(19) United States
(12) Patent Application Publication
(10) Pub. No.: US 2009/0203148 A1
(43) Pub. Date: Aug. 13, 2009

(54) Methods and Devices For Detection and Identification of Encoded Beads and Biological Molecules

(76) Inventors: Vera Gorfinkel, Stony Brook, NY (US); Boris Gorbovitski, Stony Brook, NY (US); Michael Gorbovitski, Stony Brook, NY (US)

Correspondence Address: Peter G. Carroll;Medlen & Carroll, LLP, 101 Howard Street, Suite 350, San Francisco, CA, 94105, US

(21) Appl. No.: 12/087,812

(22) PCT Filed: Jan. 19, 2007
(86) PCT No.: PCT/US07/01509

§ 371 Date: Apr. 17, 2009

Related U.S. Application Data
(60) Provisional application No. 60/760,056, filed on Jan. 19, 2006.

(51) Int. Cl. G01N 33/50; C09K 11/06; C09K 11/02; B01D 57/02; G01N 21/64

(52) U.S. Cl. 436/94; 252/301.16; 252/301.35; 204/450; 250/461.2; 977/774

(57) ABSTRACT

The invention relates to methods and devices used in the sequencing, separation, detection, and identification of objects and biological molecules. In preferred embodiments, the invention relates to a DNA sequencing system based on cyclic sequencing by synthesis which is performed on beads in three-dimensional vessels and detected using monolithic multicapillary arrays. In other embodiments, the invention relates to a bead comprising two or more luminescent labels coupled to a nucleic acid sequence. In further embodiments, said luminescent labels are quantum dots.

### RELATED APPLICATIONS

This application claims priority to U.S. provisional application No. 60/760,056 filed Jan. 20, 2006.

### FIELD OF INVENTION

The invention relates to methods and devices used in detecting, separating, and identifying encoded beads and biological molecules, and in sequencing the monomers that comprise heteropolymers. In preferred embodiments, the invention relates to a DNA sequencing system based on cyclic sequencing performed by synthesis on spectrally encoded beads, using monolith multicapillary arrays to detect synthesized products. The invention also relates to a system for performing hybridization assays on spectrally encoded beads using capillary arrays in the detection steps. The invention also relates to a system for “bar-coding” materials and objects using spectrally encoded beads and employing capillary arrays in the detection steps. In another embodiment, the invention relates to a method of creating beads assignable to mutually distinct sets, wherein each set comprises beads that each bear the same unique combination of multiple luminescent particles as labels or markers. In a further embodiment, said luminescent particles are quantum dots. In still another embodiment, the invention relates to a bead comprising two or more luminescent particles and, coupled to said bead, a nucleic acid sequence. In yet another embodiment, the invention relates to a nucleic acid coupled to a luminescent particle.

### BACKGROUND

Methods for diagnosing disease typically rely on identifying a particular biomarker, e.g., a marker indicative of the presence of mutants. However, difficulty in identifying a biomarker in the presence of the sample environment and possibly similarly structured but irrelevant biomolecules is a formidable challenge. Separation of variant nucleotide forms, for example, is often not a straightforward task, particularly if a specific variant is in a low concentration compared to a dominantly expressed version. In whole-genome DNA sequencing using hybridizing probes, one faces challenges in designing strategies that avoid cross-hybridization to the incorrect targets as a result of repetitive elements or chance similarities that contribute to a high false-positive detection rate. In addition, many methods require sample-preparation steps, as the relevant fraction of the genome must be amplified by PCR before hybridization. All in all, there is a need for novel methods that improve separation and identification of biologically distinct materials that differ only slightly in their chemical and physical properties.

### SUMMARY OF INVENTION

The invention relates to methods and devices used in detecting, separating, and identifying encoded beads and biological molecules, and in sequencing the monomeric units that comprise heteropolymers. In preferred embodiments, the invention relates to a DNA sequencing system based on cyclic sequencing performed by synthesis on spectrally encoded beads, using monolith multicapillary arrays to detect synthesized products. The invention also relates to a system for performing hybridization assays on spectrally encoded beads using capillary arrays in the detection steps. The invention also relates to a system for “bar-coding” materials and objects using spectrally encoded beads and employing capillary arrays in the detection steps. In another embodiment, the invention relates to a method of creating beads assignable to mutually distinct sets, wherein each set comprises beads that each bear the same unique combination of multiple luminescent particles as labels or markers. In a further embodiment, said luminescent particles are quantum dots. In still another embodiment, the invention relates to a bead comprising two or more luminescent particles and, coupled to said bead, a nucleic acid sequence.

In some embodiments, the invention relates to a method of generating luminescent encoded beads comprising: a) providing: i) a plurality of first identical luminescent particles, ii) a plurality of second identical luminescent particles, iii) a first plurality of porous structures, iv) a plurality of first wells, each such well containing a portion of said plurality of first luminescent particles, wherein said first luminescent particles are at different concentrations in at least two of said first wells; v) a plurality of second wells, each such well containing a portion of said plurality of second luminescent particles, wherein said second luminescent particles are at different concentrations in at least two of said second wells; b) distributing a portion of said plurality of porous structures to each of said first wells under conditions such that said first luminescent particles are absorbed by said porous structures; c) extracting said porous structures from unabsorbed first luminescent particles; d) recombining said extracted porous structures to form a second plurality of porous structures having said first luminescent particles absorbed thereon, wherein at least two of said porous structures have said particles absorbed thereon at different concentrations; e) distributing a portion of said recombined porous structures to each of said second wells under conditions such that said second luminescent particles are absorbed by said porous structures; f) extracting said porous structures from unabsorbed second luminescent particles; g) recombining said extracted porous structures to form a third plurality of porous structures having said first luminescent particles and said second luminescent particles absorbed thereon, wherein at least two of said porous structures have different concentrations of said first luminescent particles and different concentrations of said second luminescent particles. In further embodiments, said first luminescent particle is a quantum dot. In further embodiments, said second luminescent particle is a quantum dot wherein said first and second quantum dot have a different size. In further embodiments, said porous structures are mesoporous silica beads. In further embodiments, said porous structures are mesoporous polystyrene beads. In further embodiments, said conditions for distributing a portion of said plurality of porous structures to said first plurality of wells does not saturate the porous structures with said first plurality of particles.

In additional embodiments, the method further comprises providing a plurality of third identical luminescent particles apportioned among a plurality of third wells, wherein said third luminescent particles are at different concentrations in at least two of said third wells and, further, distributing a portion of said third plurality of porous structures to each of said third wells such that said third luminescent particles are absorbed by said porous structures, extracting said porous structures from unabsorbed third luminescent particles, recombining said extracted porous structures to form a fourth plurality of porous structures having said first luminescent particles, said second luminescent particles and said third luminescent particles absorbed thereon, wherein at least three of said porous structures have different combinations, by concentration, of said first, second and third luminescent particles.

In some embodiments, the invention relates to a method of determining the authenticity of an object comprising a) providing i) an object comprising a plurality of luminescent encoded beads, wherein said encoded beads comprise two or more luminescent markers configured to provide a luminescent signature, ii) electromagnetic radiation, and iii) an instrument for detecting electromagnetic radiation; b) exposing said object to said electromagnetic radiation under conditions such that said luminescent markers luminesce, and c) detecting said luminescent signature with said instrument; and d) correlating the luminescent signature with the authentic signature of said object. In further embodiments, said object is selected from the group consisting of a personal identification card, currency, liquid, solid, and fabric. In further embodiments, said electromagnetic radiation is ultraviolet light. In further embodiments, said luminescent markers are quantum dots.

In some embodiments, the invention relates to a method of generating luminescent encoded beads comprising: a) providing: i) a plurality of first luminescent particles, ii) a plurality of second luminescent particles, and iii) a plurality of porous structures; iv) a first plurality of wells wherein said first luminescent particles are in said wells and have different concentrations in at least two of the wells; v) a second plurality of wells wherein said second luminescent particles are in said wells and have different concentrations in at least two of the wells; b) distributing a portion of said plurality of porous structures to said first plurality of wells under conditions such that said first luminescent particles are absorbed by said porous structures; c) extracting said plurality of porous structures with said first luminescent particles from said first plurality of wells; d) mixing said extracted plurality of porous structures with said first luminescent particles together to form a plurality of porous structures wherein at least two of said porous structures have different concentrations of said first luminescent particles; e) distributing said plurality of porous structures wherein at least two of said porous structures have different concentrations of said first luminescent particles to said second plurality of wells under conditions such that said second luminescent particles are absorbed by said porous structures; f) extracting said plurality of porous structures with said first luminescent particles and said second luminescent particles from said second plurality of wells g) mixing said plurality of porous structures with said first luminescent particles and said second luminescent particles that are extracted from said second plurality of wells together to form a plurality of porous structures wherein at least two of said porous structures have different concentrations of said first luminescent particles and have different concentrations of said second luminescent particles. In further embodiments, said first luminescent particle is a quantum dot. In further embodiments, said second luminescent particle is a quantum dot wherein said first and second quantum dot have a different size. In further embodiments, said porous structures are mesoporous silica beads. In further embodiments, said porous structures are mesoporous polystyrene beads. In further embodiments, said conditions for distributing a portion of said plurality of porous structures to said first plurality of wells does not saturate the porous structures with said first plurality of particles.

In additional embodiments, the method further comprises providing a plurality of third luminescent particles, wherein said second and third luminescent particles are in said wells and have different concentrations in at least two of the wells and further mixing said plurality of porous structures with said first luminescent particles said second luminescent particles and said third luminescent particles that are extracted from said second plurality of wells together to form a plurality of porous structures wherein at least three of said porous structures have different combinations of concentrations of said first, said second and said third luminescent particles.

In some embodiments, the invention relates to a method of determining the authenticity of an object comprising a) providing i) an object comprising plurality luminescent encoded beads, wherein said encoded beads comprise two or more luminescent markers configured to provide a luminescent signature, ii) electromagnetic radiation, and iii) an instrument for detecting electromagnetic radiation; b) placing said object in said electromagnetic radiation under conditions such that said quantum dots luminesce, and c) detecting said luminescent signature with said instrument; and d) correlating the luminescent signature with the authenticity of said object. In further embodiments, said object is selected from the group consisting of a personal identification card, cash, liquid, solid, and fabric. In further embodiments, said electromagnetic radiation is ultraviolet light. In further embodiments, said luminescent markers are quantum dots.

In further embodiments, the invention relates to a method of moving a bead through a channel comprising: a) providing: i) bead comprising a first luminescent label and a second luminescent label, ii) a channel, iii) a solution inside said channel wherein said beads are inside said solution, iv) pair of electrodes; and b) applying a potential between said pair of electrodes under conditions such that said bead moves in said channel toward one electrode of said electrode pair. In further embodiments, said bead is a polystyrene bead. In further embodiment, said first and second luminescent labels are quantum dots. In further embodiments, said bead is charged.

In some embodiment, the invention relates to a method of determining a phenotype of a subject comprising: a) providing, i) a plurality of linked beads wherein said beads comprise: A) luminescent electromagnetic codes, B) a plurality of nucleic acid markers which hybridize to nucleic acids that correlate to a phenotype of a subject, and wherein said plurality of nucleic acids markers are configured such that nucleic acids with a unique sequence are linked to a bead with a unique luminescent electromagnetic code, and ii) a sample containing or suspected of containing nucleic acid from said subject; b) detecting said luminescent electromagnetic codes on said plurality of beads and recording said codes to correspond to said unique sequence of said nucleic acid marker on said beads; c) mixing said linked beads with said sample under conditions such that hybridization to nucleic acids in said sample can occur; d) detecting a bead where hybridization occurs; e) determining said luminescent electromagnetic code on said hybridized bead; f) comparing said luminescent electromagnetic code on said hybridized bead to said recorded codes; and g) correlating said recorded code to said phenotype in said subject. In some embodiments, said luminescent electromagnetic codes comprises more than three distinguishable electromagnetic wavelengths. In some embodiments, said electromagnetic wavelengths are discrete visual colors. In some embodiments, said beads are linked to said nucleic acids by a biotin-streptavidin interaction. In some embodiments, said phenotype is a disease. In some embodiments, said subject is a human. In further embodiments, a number of said beads exceeds 1,000,000 or 10,000,000. In further embodiments, said plurality of nucleic acid markers includes 1000 or 10,000 different markers.

In some embodiments, the invention relates to a method comprising: a) providing: i) a bead comprising a first luminescent label and a second luminescent label, ii) a first nucleic acid, iii) a second nucleic acid, a portion of the nucleotide sequence of which is complementary to a portion of the nucleotide sequence comprising said first nucleic acid, iv) a nucleotide comprising a third luminescent label, and v) a transparent channel; b) attaching said first nucleic acid to said bead; c) contacting said second nucleic acid and said first nucleic acid under conditions such that said contacting results in the formation of a double stranded portion of the first nucleic acid; d) mixing said nucleotide and said double stranded portion under conditions such that ligation of said nucleotide to said second nucleic acid provides a ligated nucleic acid; e) moving said bead through said transparent channel; and f) detecting independently said first, second and third luminescent labels. In further embodiments, the method comprises the additional step of g) removing the third luminescent label from said ligated nucleic acid. In further embodiments, the method comprises repeating steps d)-g). In further embodiments, said first and second luminescent labels are contained in the bead. In further embodiments, said first and second luminescent labels are covalently attached to the exterior of the bead. In further embodiments, said first and second luminescent labels are quantum dots capable of fluorescing. In further embodiments, said first luminescent label is a dye and said second fluorescent label is a quantum dot. In further embodiments, said first and second luminescent label are dyes. In further embodiments, said nucleotide is a nucleotide triphosphate. In further embodiments, said bead comprises different concentrations of said first and second luminescent label. In further embodiments said different concentrations are in an amount of label per bead, amount of label per unit volume of bead, or amount of label per volume of a solution in which the bead is suspended.

In another embodiment, the invention relates to a detection system comprising: a) a first bead comprising a first luminescent label and a second luminescent label; b) a second bead comprising a third luminescent label and a fourth luminescent label; c) a first transparent channel comprising said first bead and a second transparent channel comprising said second bead; and d) an instrument for detecting electromagnetic radiation from luminescent labels. In further embodiments, said first and second luminescent labels are contained in the bead. In further embodiments, said first and second luminescent labels are covalently attached to the exterior of the bead. In further embodiments, said first and second luminescent labels are fluorescent quantum dots. In further embodiments, said first luminescent label and said third luminescent label are the same label wherein said third luminescent label is at lower concentration inside said second bead than the concentration of said first luminescent label in said first bead. In further embodiments, a common wall separates said first and second transparent channels. In further embodiments, said first and second transparent channels comprise a square cross section. In further embodiments, said instrument for detecting electromagnetic radiation is a charge-coupled device. In further embodiments the system comprises a source of electromagnetic radiation. In further embodiments, said source of electromagnetic radiation is a laser.

In other embodiments, the invention relates to a detection system comprising: a) a first bead comprising a first luminescent label, a second luminescent label and a first nucleic acid comprising a first nucleotide sequence having a first removable luminescent marker on the last nucleotide in said sequence; b) a second bead comprising a third luminescent label, a fourth luminescent label and a second nucleic acid comprising a second nucleotide sequence having a second removable luminescent marker on the last nucleotide in said sequence; c) a first transparent channel configured to accept said first bead and a second transparent channel configured to accept said second bead; d) an instrument for detecting electromagnetic radiation from said luminescent labels; and e) an instrument for detecting electromagnetic radiation from said removable luminescent markers. In further embodiments said instrument is configured to collect separate datasets for each luminescent label. In further embodiments, the system comprises a dichroic mirror. In further embodiments, said first and second luminescent labels are contained in the bead. In further embodiments, said first and second luminescent labels are fluorescent quantum dots. In further embodiments, said removable luminescent marker is removable upon exposure to light. In further embodiments, said removable luminescent marker is linked to said nucleotide by an ortho nitrophenyl group. In further embodiments, said instrument for detecting electromagnetic radiation from said luminescent labels comprises a charge-coupled device. In further embodiments, said instrument for detecting electromagnetic radiation from said luminescent marker comprises a charge-coupled device. In further embodiments, the system further comprises a laser. In further embodiments, said laser shines electromagnetic radiation into said first and second transparent channels. In further embodiments, the system comprises a pump configured to reversibly push said first and second beads through said transparent channels.

In another embodiment, the invention relates to a method of determining the nucleotide sequence of a nucleic acid comprising: a) providing: i) a detection system comprising: (A) a first bead comprising a first luminescent label and a second luminescent label; (B) a second bead comprising a third luminescent label and a fourth luminescent label; C) a first transparent channel and a second transparent channel; and D) an instrument that simultaneously projects electromagnetic radiation into said first transparent channel and said second transparent channel; ii) a first nucleic acid and a second nucleic acid wherein said first and second nucleic acid have identical or complementary overlapping nucleotide sequences; iii) a plurality of primers that hybridize to one end of said first and second nucleic acids; iv) a set of nucleotides comprising removable luminescent markers wherein luminescence of each of said markers corresponds to a unique nucleoside base; b) coupling said plurality of primers to said first bead and said second bead; c) contacting said first bead and said first nucleic acid under conditions such that hybridization of said first nucleic acid to one of said primers occurs and contacting said second bead and said second nucleic acid under conditions such that hybridization of said second nucleic acid to one of said primers occurs; d) exposing said set of nucleotides to said first and second beads under conditions such that said nucleotides ligate to said primers in accordance with hydrogen bonding pairing of a corresponding nucleoside base on said hybridized first and second nucleic acids; e) placing said first bead in said first transparent channel and said second bead in said second transparent channel such that said projected electromagnetic radiation illuminates said labels and markers; and d) detecting said labels and markers to correspond to first and second nucleic acid sequence coupled to said first and second beads. In further embodiments, the method comprises removing said marker from said ligated nucleotide. In further embodiments, the method comprises repeating steps d)-f). In further embodiments, said primers comprise in whole or in part, an identical nucleotide sequence. In further embodiments, said first and second nucleic acids comprise a nucleotide sequence complementary to said primers. In further embodiments, said coupling occurs by said beads comprising streptavidin and said primers comprising biotin.

In another embodiment, the invention relates to a method of generating luminescent encoded beads comprising: a) providing: i) a plurality of first luminescent particles a plurality of second luminescent particles, ii) a plurality of porous structures; iii) a first plurality of wells; wherein said first luminescent particles are in said wells and have different concentrations in at least two of the wells; and iv) a second plurality of wells wherein said first luminescent particles are in said wells and have different concentrations in at least two of the wells, b) distributing a portion of said plurality of porous structures to said first plurality of wells under conditions such that said first luminescent particles are absorbed by said porous structures, c) mixing said plurality of porous structures with said first luminescent particles that are in said first plurality of wells together to form a plurality of porous structures wherein at least two of said porous structures have different concentrations of said first luminescent particles, d) distributing said plurality of porous structures wherein at least two of said porous structures have different concentrations of said first luminescent particles to said second plurality of wells under conditions such that said second luminescent particles are contained in said porous structures; and e) mixing said plurality of porous structures with said first luminescent particles and said second luminescent particle that are in said second plurality of wells together to form a plurality of porous structures wherein at least two of said porous structures have different concentrations of particles per bead of said first luminescent particles and have different concentrations of said second luminescent particles. In further embodiments, said first luminescent particle is a quantum dot. In further embodiments, said second luminescent particle is a quantum dot wherein said first and second quantum dot have a different size. In further embodiments, said porous structures are mesoporous silica beads. In further embodiments, said conditions for distributing a portion of said plurality of porous structures to said first plurality of wells does not saturate the porous structures with said first plurality of particles.

In some embodiments, the invention relates to particles containing two or more different fluorophores are modified in a manner to comprise biomolecules. In further embodiments, the fluorophores are quantum dots and the biomolecules are nucleic acids. And in further embodiments, the particles are mixed with a sample that contains or is suspected of containing a nucleic acid having a complementary nucleotide sequence to alt least one of the nucleotides comprised in said particle and are manipulated in a manner to determine a nucleic acid sequence. The particle is subject to movement through a capillary array and the hybridized nucleic acid sequence is identified by the fluorescent emission of the quantum dots.

In some embodiments, the invention relates to a method of identifying a specific molecule comprising: a) providing i) a sample suspected of having a first molecule and ii) a bead conjugated to a second molecule wherein said bead comprises a first optical marker and a second optical marker; b) mixing said sample and said bead under conditions such that said first molecule binds to said second molecule forming a conjugate complex; c) separating said bead from said sample under conditions that said conjugate complex is purified; and d) detecting said first and second optical markers. In further embodiments, said first molecule is a first nucleic acid, amino acid sequence or polysaccharide. In further embodiments, said second molecule is a second nucleic acid with a complementary sequence to a portion of said first nucleic acid. In further embodiments, said binding is by hybridization of said first nucleic acid to said second nucleic acid. In further embodiments, said conjugate complex is a double-stranded nucleic acid. In further embodiments, said first optical marker is a quantum dot. In further embodiments, said second optical marker is a quantum dot. In further embodiments, said separating conditions are by capillary electrophoresis. In further embodiments, said bead comprises said first optical marker in a higher concentration than said second optical marker. In other embodiments, the invention relates to a method of identifying a specific molecule comprising: a) providing i) a sample suspected of having a first molecule, ii) a first bead conjugated to a second molecule wherein said first bead comprises a first optical marker and a second optical marker, and iii) a second bead conjugated to a third molecule wherein said second bead comprises a first optical marker and a second optical marker wherein said first optical marker in said second bead is in a higher concentration than in said first bead; b) mixing said sample and said first and second beads under conditions such that said first molecule is capable of binding to said second molecule forming a conjugate complex; c) separating said first and second bead from said sample under conditions such that said conjugate complex is purified; and d) detecting said first and second optical markers. In further embodiments, said first molecule is a first nucleic acid, amino acid sequence or polysaccharide. In further embodiments, said second molecule is a nucleic acid complementary sequence to a portion of said first nucleic acid. In further embodiments, said binding is hybridization of said first nucleic acid to said second nucleic acid. In further embodiments, said conjugate complex is a double-stranded nucleic acid sequence. In further embodiments, said first optical marker is a quantum dot. In further embodiments, said second optical marker is a quantum dot. In further embodiments, said separating condition is by capillary action.

In some embodiments, the invention relates to a method of detection and identification of encoded beads in capillary array comprising: a) providing i) a plurality of beads preferably of a size of less than 10 μm or even more preferably of less than 1 μm, and even more preferred, the beads contain pores between 10 and 30 nanometers, diluted in a buffer to a desired concentration wherein each bead carries a unique code and can be identified by this code; ii) a container for holding the diluted set of beads; iii) a multi-capillary array; iv) a pumping instrument for moving said beads from said container through the capillary array; v) an excitation instrument for exciting a signal from the beads; a detection instrument for acquiring signals from said beads while they are passing through said capillaries; vi) an instrument for transferring and recording detection data; and vii) an instrument for processing said data; b) pumping the set of beads from the container through the multi-capillary array; c) exciting the beads with said excitation instrument under conditions such that a signal is generated by said bead; d) detecting said signal with said detection instrument and e) processing said signals with said processing instrument. In further embodiments, said binding includes epitope binding of an antibody. In further embodiments said antibody is bound to said bead and said epitope is bound to a cell. In further embodiments, said processing instrument is a computer. In further embodiments, said beads are encoded spectrally. In further embodiment, the spectral encoding is digital, analog or both. In further embodiments, each bead carries an additional color marker which differs from encoding color markers and which signals the presence of the beads in a bead detection region of the capillary. In further embodiments, the beads are detected by illumination induced fluorescence. In further embodiments, the beads are microspheres encoded with multi-color quantum dots. In further embodiments, the beads are mesoporous. In further embodiments, the capillary array is a monolithic glass structure with holes of arbitrary shape. In further embodiments, said capillary array contains more than 2 capillaries arranged in a row i.e., a linear array. In further embodiments, said capillary array is arranged in a two-dimensional cross section. In further embodiments, said capillary array is fabricated by a glass pulling process or by gluing together individual capillaries. In further embodiments, the bead detection system detects the beads simultaneously or sequentially, preferably in a scanning fashion, in all capillaries of the capillary array. In further embodiments, the detection system detects beads in a plane perpendicular to the capillaries of the array. In further embodiments, the detection system detects beads in a plane that crosses the capillaries under a certain angle to the capillaries of the array by the side.

In some embodiments, the invention relates to a method for the detection and identification of biomolecules using encoded beads in a capillary array comprising: a) providing i) a plurality of beads preferably of a size of less than 10 μm or even more preferably of less than 1 μm, diluted in a buffer to a concentration wherein each bead carries a unique code and can be identified by this code and wherein each bead is covered with a specific biomolecule which selectively binds, or preferably hybridizes to said biomolecules to be identified; ii) a set of biomolecules to be identified, iii) a container for holding the diluted set of beads and biomolecules in a buffer; iv) a multi-capillary array; v) a pumping instrument for moving said beads from said container through the capillary array; vi) an excitation instrument for exciting a signal from the beads which carries information about the codes of the beads as well as information on binding of biomolecules to the beads; vii) a detection instrument for encoding signals from said beads detecting binding of said biomolecules to correspond to said beads while they are passing through said capillaries; viii) an instrument for transferring and recording detection data; and ix) an instrument for processing said data which are able to identify codes of every individual bead; b) pumping the set of beads from the container through the multi-capillary array; c) exciting the beads with said excitation instrument under conditions such that a signal is generated in said bead; d) detecting said signal with said detection instrument and e) processing said signals with said instruments.

In other embodiments, the invention relates to a method of the detection and identification of biomolecules using encoded beads in a capillary array comprising: a) providing i) a plurality of beads preferably of a size of less than 10 μm or even more preferably of less than 1 μm, diluted in a buffer to a desired concentration wherein each bead carries a unique code and can be identified by this code and wherein each bead is covered with a specific biomolecule which selectively binds, or preferably hybridizes to said biomolecules to be identified; ii) a set of biomolecules to be identified, iii) a set of chemical reagents to carry out biological reactions, preferably PCR and cycle sequencing iv) a container for holding the diluted set of beads, a set of chemical reagents and biomolecules in a buffer, v) a multi-capillary array with at least one capillary; vi) a pumping instrument for moving said beads from said container through the capillary array; vii) an excitation instrument for exciting a signal from the beads which carries information about the codes of the beads as well as information on binding of biomolecules to the beads, viii) a detection instrument for encoding signals from said beads detecting binding of said biomolecules to correspond to said beads while they are passing through said capillaries; ix) an instrument for transferring and recording detection data; and x) an instrument for processing said data which are able to identify codes of every individual bead; b) pumping the set of beads from the container through the multi-capillary array; c) exciting the beads with said excitation instrument under conditions such that a signal is generated in said bead; d) detecting said signal with said detection instrument and e) processing said signals with said instruments under conditions such that one is able to identify codes for every individual bead. In further embodiments, said sequence of biological reactions includes cycle sequencing and said sequencing of chemical reactions and bead detection and identification are repeated which allow sequencing for nucleic acid sequences. In further embodiments, the invention relates to devices that perform the detection of beads disclosed herein.

In some embodiments, the invention relates to a DNA sequencing system using cyclic sequencing by synthesis method that is performed on beads in three-dimensional vessels and using monolith multi-capillary arrays for separation of the beads.

In some embodiments, the invention relates to addressable beads working all at once to search through a forest of nucleic acids until each bead finds its quarry, unless its quarry isn't there, and then each bead goes through analysis where one identifies the beads and determines whether or not the beads found what they went to find.

In some embodiments, the invention relates to a nanometer scale PCR system for quantitative analysis of molecular markers for cancer. In further embodiments said markers are telomerase repeats. In further preferred embodiments, said markers are fluorescently labeled.

In further embodiments, the invention relates to single molecule amplification in capillaries filled with alternating nanoliter scale zones of PCR reagents. In further embodiments the zones alternated with a zone of aqueous solutions of PCR reagents and a zone of oil.

In another embodiment, the invention relates to a method comprising providing a DNA library on encoded beads, sequencing by synthesis on the individual beads following by bead flow and detection in a multicapillary array.

In further embodiments, the method comprises preparing a DNA library on spectrally encoded beads; incubating the beads with a labeled nucleotide, e.g., A; detecting the encoded bead with the incorporated labeled nucleotide using a multi-capillary array; detaching fluorescent labels from incorporated nucleotides; and repeating the steps using another nucleotide, e.g., A, T, C, G, U.

In further embodiments, the beads are pumped from the tube through a multi-color illumination multi-capillary array after every incubation cycle with labeled nucleotides. Detection of individual beads are done in real time using a laser or light emitting illumination source for fluorescence excitation and a CCD camera.

In some embodiments, each bead carries a distinct spectral code so that specific sequences can be related to individual beads even though a spatial position of the beads may change. Parallel sequence detection is performed by pushing the beads through a glass monolith multi-capillary array with consists of k×1 square capillaries, e.g., 100×100, which are 2-5 μm inner diameter, 5-10 μm pitch, 2-3 cm capillary length.

In a preferred embodiment, the beads carry 10⁶ to 10⁹ distinct spectral codes. In another preferred embodiments, the monolith multi-capillary arrays have 1,000 to 100,000 capillaries.

In another preferred embodiment, an optical detection system is capable of detecting of up to 10 colors with 2 μm resolution in an area of up to 1 cm².

In some embodiments, the invention relates to the used of beads that are optically coded using segmented nanorods, rare-earth doped glass, fluorescent silica colloids, photobleached patterns, oligonucleotide linked colloidal gold, or enhanced Raman nanoparticles.

In preferred embodiments, luminescent quantum dots are used. In an even more preferred embodiment, mesoporous polystyrene beads encoded with surfactant-coated quantum dots that can be identified using a flow cytometer at a readout out of up to 1000, 5000, 10,000, 50,0000, 100,000, 500,000, 1,000,000, or 10,000,000 beads per second.

In some embodiments, the invention relates to nanocrystals of quantum dots with a multitude of various sizes within the nanocrystal core, i.e., quantum dots of a plurality of discrete sizes are mixed and coated within a shell. Because a quantum dot of small size provides a specific fluorescence emission different from the fluorescent emission of a larger quantum dot, a nanocrystal containing a mixture of small and large quantum dots will result in multiple fluorescent signals upon excitation.

In addition, in some embodiments, the invention relates to nanocrystals where the relative number of the small or large quantum dots can be adjusted in order to intensify or decrease the extent of the fluorescent signal at a specific wavelength.

In certain embodiments, the invention relates to tracking specific modifications of a nanocrystal with a specific quantum dot makeup to the existence of a particular biomolecule linked to the exterior. The biomolecule linked to the exterior of the nanocrystal may be exposed to a composition containing binding molecules.

In further embodiments, the biomolecules are nucleic acid sequences that hybridized to specific complimentary sequences.

In certain embodiments, the invention relates to providing a plurality of nanocrystals corresponding to a plurality of nanocrystal cores containing a plurality of sizes of quantum dots and a plurality of the number of quantum dots of a specific size.

In some embodiments, the invention relates to a system based on hybridization of biomolecules or cells to multicolor beads that have distinct color signatures and carry specific genetic probes.

In certain embodiments, it is not intended that the claims be limited by the method of encoding the beads with color. There are various methods of encoding beads with multiple colors such as adding molecular dyes to a particle.

In a preferred embodiment, micrometer scale beads contain multicolor quantum dots. It is not intended that the fluorescent emission of the quantum dots be limited to visible light.

In preferred embodiments, the fluorescent emission comprises a blue color. In other embodiments, the fluorescent emission is infrared.

In some embodiments, the encoding signal may be digital, e.g., the encoding color is either present or absent.

In some embodiments, encoding signal may be analog, i.e., measure of the relative emission intensity. This may be done for each individual color.

In some embodiments, the invention relates to a method of using a set of encoded beads coated with specific molecular probes in hybridization assay in a single tube format. Hybridization with encoded beads is done by a spectral coding method. If N number of colors is used, then 2^{N} distinct color combinations can be identified. If N numbers of colors are used and M numbers of intensity resolution frequencies are used, then 2^{NM} distinct color combinations can be identified. For example, 65,000 unique beads can be encoded using either 16 colors or 4 colors with 4 different intensity resolutions. After hybridization, the sample can be pumped into a three-dimensional multichannel analyzer. One may detect individual beads in real time using a laser or other light-emitting source such as a light emitting diode. Detection of the bead flow may be done with a digital camera either from the top or from the side of the multichannel analyzer. The detected signal, digital or analog, is then transferred to a computer for storage and analysis.

In another embodiments, the invention relates to a capillary array fabrication system comprising a ingot for shaping the capillaries having a feed segment, a heater, an area for holding a solution for coating the inside cavities and outside portion of the monolith, lamps, preferable ultraviolet lamps for curing the monolith, and rollers for moving the monolith.

In other embodiments, the invention relates to beads comprising antibodies wherein said bead have a plurality of luminescent markers. In further embodiments, the antibodies bind amino acid sequences that are incorporated into nucleotides.

In further embodiments, the invention relates to nucleotides conjugated to amino acid sequences linked by photodegradable moiety wherein said amino acid sequences will bind to antibodies conjugated to beads with a plurality of luminescent markers preferably quantum dots.

In further embodiments, the invention relates to sequencing nucleic acids using bead comprising antibodies with a plurality of luminescent markers.

In further embodiments, the invention relates to a method of detecting or sequencing a nucleic acid by using nucleotides conjugated to an amino acid sequence.

In further preferred embodiments, the nucleic acid is linked by a photodegradable moiety, and in a further embodiment, said amino acid sequence is the epitope for an antibody conjugated to a bead comprising quantum dots.

In some embodiments, the invention relates to a method of incorporating a nucleotide into a growing double stranded nucleic acid comprising mixing a nucleic acid and an nucleotide conjugated to a marker, preferably the marker is an amino acid sequence conjugated with a photodegradable linker, under conditions such that said nucleotide hybridizes to a complimentary base and ligates to the growing strand of the nucleic acid sequence; mixing the nucleic acid sequence with the incorporated nucleotide with an antibody having a specific binding of an epitope to said amino acid sequence conjugate to the nucleotide, wherein said antibody is conjugated to a luminescent marker preferably a bead comprising a quantum dot; measuring said antibody luminescent marker; and correlating said marker to the incorporated/ligated nucleotide.

In further embodiments, said nucleic acid is conjugated to a solid support. In further embodiments, the support is an array where the content of the nucleic acid sequence is being correlated to the position in the array.

In other embodiments, the invention relates to method of manipulating nucleic acid sequences and nucleotides using compositions and instruments disclosed herein.

In additional embodiment, the invention relates to the use of spectrally encoded beads in document authenticity methods.

In some embodiments, the invention relates to a method of determining the authenticity of a document comprising a) providing i) a document comprising plurality encoded beads, wherein said encoded beads comprise two or more luminescent markers configured to provide a luminescent signature, ii) electromagnetic radiation, and iii) an instrument for detecting electromagnetic radiation; b) placing said document in said electromagnetic radiation under conditions such that said quantum dots luminesce, and c) detecting said luminescent signature with said instrument; and d) correlating the luminescent signature with the authenticity of said document. In further embodiments, said document is a certified check. In further embodiments, said document is cash. In further embodiments, said electromagnetic radiation is ultraviolet light. In further embodiments, said luminescent markers are quantum dots.

In some embodiments, the invention relates to a method of moving a bead through a channel comprising: a) providing: i) bead comprising a first luminescent label and a second luminescent label, ii) a channel, iii) a solution inside said channel wherein said beads are inside said solution, iv) pair of electrodes; and b) applying a potential between said pair of electrodes under conditions such that said bead moves in said channel toward one electrode of said electrode pair. In further embodiments, said bead is a porous polystyrene bead. In further embodiments, said first and second luminescent labels are quantum dots. In further embodiments, said bead is charged. In further embodiments, said bead has a carboxyl functionalized surface.

### BRIEF DESCRIPTION OF THE DRAWINGS

FIG. 1 illustrates a preparation of a bead with luminescent markers and conjugation of nucleic acid sequences of disease markers.

FIG. 2 illustrates a schematic of the DNA sequencing method. The method comprises the following steps: preparation DNA library on spectrally encoded beads; incubation of beads with labeled nucleotides (e.g. A); section of spectrally encoded beads with incorporated labeled nucleotides in the MMCA using micro-pump; detaching fluorescent labels form incorporated nucleotides; and addition of the next nucleotide and repetition of all steps. Each bead carries a distinct spectral code so that specific sequences can be related to individual beads even though a spatial position of the beads may change. Highly parallel sequence detection is performed by pushing the beads through a glass monolith multi-capillary array which consists of k×1 square capillaries (e.g. 100×100).

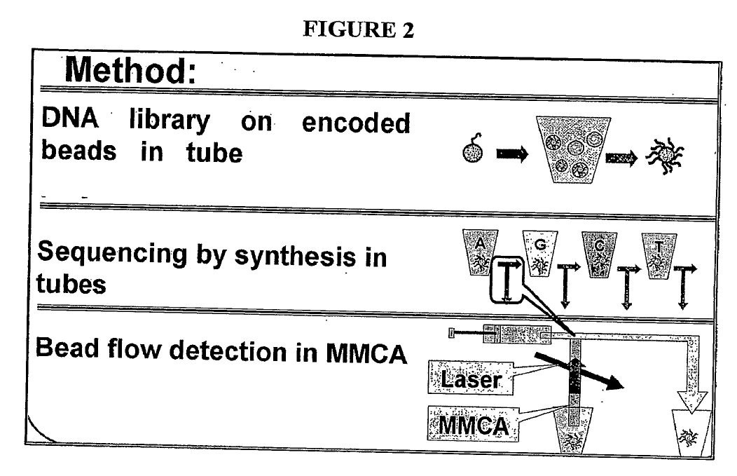

FIG. 3 illustrates a use of a multicapillary array in synthesis and detection methods. The beads are pumped from the tube through the monolith multi-capillary array (MMCA). Detection of individual beads is done from the top of the MMCA in real time fashion using laser or LED illumination source for fluorescence excitation and fast CCD cameras.

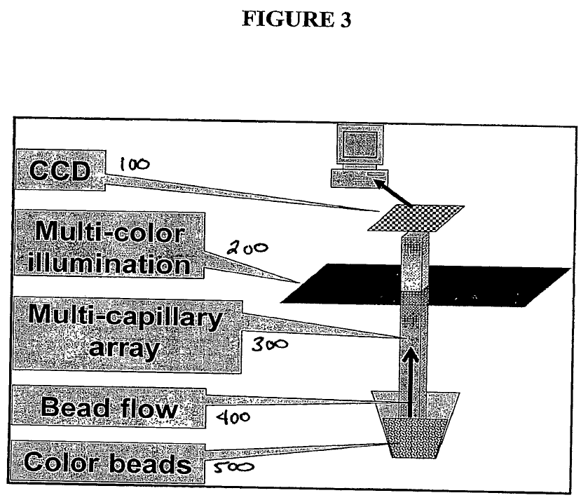

FIG. 4 illustrates a method of creating beads with multiple colors and gradations. A large amount of M colorless porous beads (M>>10⁹) is distributed between 10 wells filled with solutions of different concentration of the first type of quantum dot (QD₁). After embedding QD₁ into the beads, contents of all 10 wells are mixed together, the beads are washed, and randomly distributed between the next set of 10 wells filled with different concentrations of QD₂. The procedure is repeated 9 times and after the 9^{th} cycle, one obtains a set of M beads that carry all possible combinations of 10⁹ color codes. In another embodiment, the invention relates to a method wherein a large amount of M colorless porous beads (M>>10⁹) is distributed between 25 wells filled with solutions of mixtures of QD₁ and QD₂ in different concentrations. After embedding quantum dots into the beads, contents of all 25 wells are mixed together, the beads are washed, and randomly distributed between the next set of 20 wells filled with different concentrations of QD₃+QD₄. The procedure is repeated T times adding new sets of wells with various concentrations of different quantum dots. After the T^{th} cycle, one obtains a set of M beads that carry all possible combinations of color codes.

FIG. 5 illustrates a preferred embodiment for a bead identification system having a laser illuminate the beads that are present and detecting the illuminated beads with a plurality of CCD detectors.

FIG. 6 illustrates a preferred embodiment of a fluidic bead transfer system with the monolith multicapillary array (MMCA).

FIG. 7 illustrates the fabrication of MMCA. The MMCAs are fabricated from ingots and ferrules which are heated into the array as part of a pulling process, see Example 5

FIG. 8 shows an eletropherogram corresponding to detected beads flowing through a capillary channel. Streptavidin-coated polystrene 2 μm beads were labeled with fluorescein using incubation with biotinylated antibody followed by the incubation with fluorescence-conjugated antibody.

FIG. 9 shows a photograph of the linear MMCA with square holes having 32 channels within 3 millimeters.

FIG. 10 shows a photograph of the cross section of the glass MMCA with square holes having a 32 by 24 array with a total of 728 channels.

FIG. 11 illustrates exemplary nucleotides with fluorescent markers for use in nucleic acid sequencing and detection disclosed herein.

FIG. 12 illustrates an exemplary method of making the nucleotides described in

FIG. 13 illustrates an exemplary method of nucleic acid detection using a nucleotide with a marker recognized by an antibody attached to a luminescent bead and incorporating the nucleotide into a growing strand of a nucleic acid.

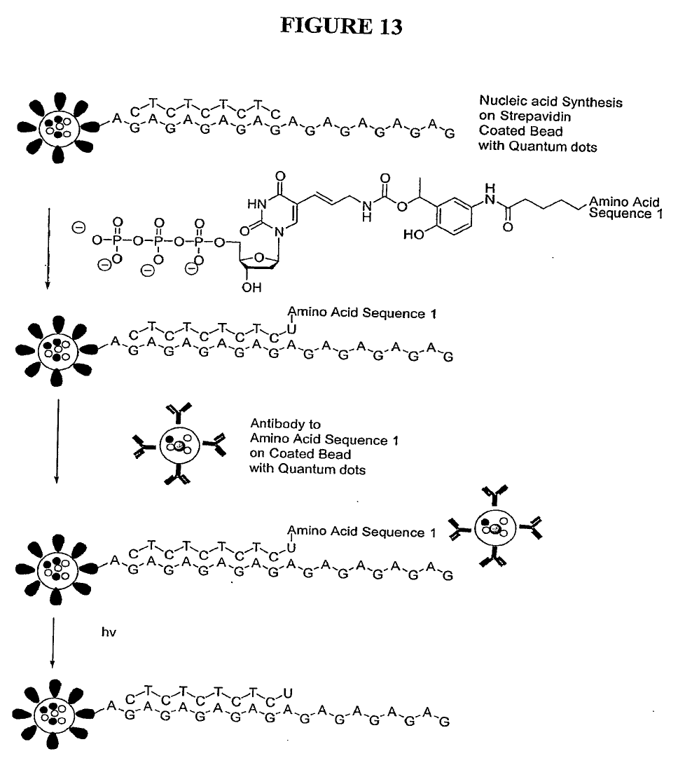

FIG. 14 illustrates a method of using beads with nucleic acid markers.

FIG. 15 illustrates a single capillary bead reader.

FIG. 16 illustrates transfer of beads in capillary using electric field (Example 10).

### LIST OF FIGURE LABELS

100 CCD
200 Multi-color illumination
300 Monolith Multi-capillary array (MMCA)
400 Bead Flow
500 Color Beads
700 Beads moving in the direction of arrow
800 Laser
900 Dichroic mirror
1000 CCD
1100 Mirror
1200 Computer (PC)
1300 Data Processor (Computer for data processing)
1500 Syringe 1
1600 Manifold 1
1700 Reservoir 1
1800 Manifold 2
1900 Manifold 3
2000 Syringe 2
2300 Reservoir 2
2400 Direction of Beads' during detection
2500 Direction of Beads during return
2600 Illumination system (laser)
2700 MMCA ingot
2800 Ingot Feed
2900 heater
3000 MMCA Coating
3100 UV Lamps
3200 Rollers
3300 Laser beam
3400 Capillary
3500 Bead
3600 Fluorescence
3700 Prism
4000 Well 1 with first electrode +(−)
4100 Well 2 with second electrode −(+)
4200 Capillary
4300 Bead

### DETAILED DESCRIPTION OF INVENTION

The invention relates to methods and devices used in separating, detecting, and identifying biological molecules and, if heteropolymeric, sequencing them. In a preferred embodiment, the invention relates to a DNA sequencing system based on cyclic sequencing by synthesis performed on beads constrained in three-dimensional vessels. The beads are detected as they pass through monolithic multicapillary arrays. In another embodiment, the invention relates to a bead comprising two or more luminescent labels coupled to a nucleic acid. In a further embodiment, said luminescent labels are quantum dots.

The disclosed DNA sequencing systems allow significant advances in means for determining the etiology of human diseases and for preventing, diagnosing and treating them including comparative profiling of tumors and tumor subtypes versus normal subtypes to identify genetic bases of malignancies; genomic profiling of immune, cardiovascular, nervous, and other systems in normal and pathological conditions; genome wide expression analysis in functional genomics; genomic identification of pathogenic microbes and detailed annotation of drug resistant strains, and contiguous sequencing of individual human genomes as an element of individual health care.

As used herein, a “channel” means a volume bounded in part by a solute-impermeable material. The channel is often used to hold a liquid or a solid or liquid suspension. It is not intended that the channels be of any specific shape. However, in preferred embodiments, the channels are shaped as cylinders. In an even more preferred embodiment, the channels are capillaries.

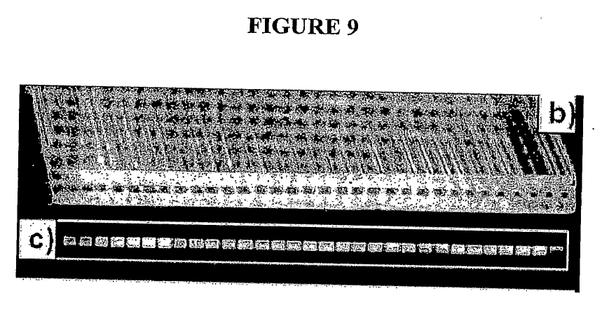

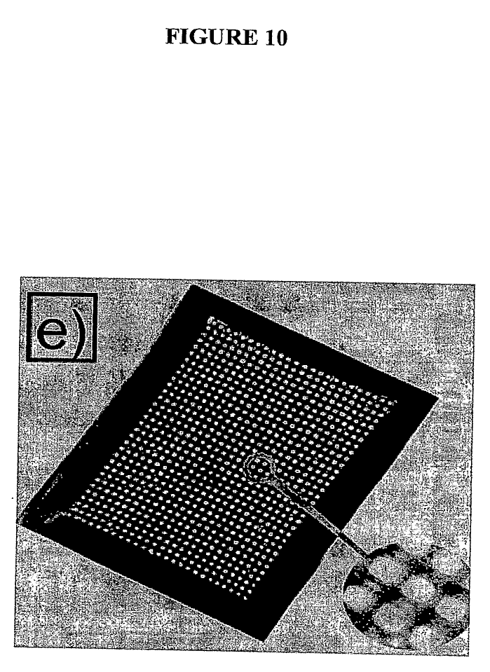

As used herein a “capillary” means a channel of sufficiently small dimension to permit capillarity to act on materials in the channel. In other preferred embodiments, the channel is made of a material that is transparent. A capillary array or multi-capillary array is a group of two or more capillaries. Examples are provided in FIGS. 9 and 10. Capillary action or capillarity or capillary motion occurs when the adhesive intermolecular forces between the gas-liquid interface of a liquid in a tube and the inner surface of the wall of the tube at that interface exceed the cohesive intermolecular forces between the gas-liquid interface and the liquid beneath that interface. Under these circumstances, a tube tends to move a liquid within it such that a gas within it is displaced. This tube is typically referred to as a capillary tube.

As used herein, the term “transparent” in reference to a material means a material through which electromagnetic radiation, preferably, but not limited to, visible light, can pass. A transparent channel is intended to mean transparent to the extent that the channel needs to be illuminated or needs to pass light and emit light to a detector for the proper functioning of the device in which it is a part. With regard to materials such as plastic or glass that are transparent, it is not intended that all electromagnetic radiation pass through the material. For example, a material that filters, reflects or absorbs certain visible wavelengths is still considered transparent.

As used herein, a “bead” means a material with a periphery of preferably less than centimeters and greater than 300 nanometers in area. Preferably the bead is substantially spherical. The bead could also be shaped in a rod or cube, but it is not intended that the bead be limited to these shapes. Preferably the bead is made of a material that is stable to dissolution in the liquid in which it is to be suspended. Preferably the bead is made of a polymer or metal or a combination thereof, but it is not intended that the bead be limited to these materials. It is contemplated that the exterior surface of the bead may vary chemically from its internal chemical constitution. It is also contemplated that the interior of the bead may have pores that contain materials that are not part of the chemical constitution of the bead itself. Reigler et al. Analytical and Bioanalytical Chemistry 384(3): 645-650 (2006) discusses coded polymer beads for fluorescence multiplexing including how to make polystyrene beads swollen with different types of nanocrystals.

Physical separation of DNA from the small beads preferred is facilitated by placing the DNA-bead mixture in an electric field between a pair of electrodes, DNA can be made to migrate to one electrode faster than the beads, thus effecting a clear separation.

In some embodiments, the invention relates to moving beads using an electropotenial. It has been discovered that carboxyl functionalized, 500 nm polystyrene divinylbenzene beads doped with Quantum dots (CrystalPlex Plex) can be moved in a capillary channel by using electrodes. It is contemplated that other charged beads such as amine functionalized beads may also be moved in a electric field.

As used herein, the term “solid support” is used in reference to any solid or stationary material to which reagents such as antibodies, antigens, and other test components are attached. For example, in the ELISA method, the wells of microtiter plates provide solid supports. Other examples of solid supports include microscope slides, coverslips, beads, particles, cell culture flasks, as well and any other suitable item.

As used herein, the term “well” means a container or reservoir to hold a liquid. It is not intended that the well be limited to any particular shape.

A “label” is a composition detectable from background by its properties, including without limitation spectroscopic, photochemical, biochemical, immunochemical, and chemical. For example, useful labels include fluorescent proteins such as green, yellow, red or blue fluorescent proteins, ³²P, fluorescent dyes, electron-dense reagents, enzymes (e.g., as commonly used in an ELISA), biotin, digoxigenin, or haptens and proteins for which antisera or monoclonal antibodies are available.

The term “different concentrations” with relation to labels and markers in or on beads mean the amount of label per bead.

Luminescence is a property of certain materials that renders them capable of absorbing electromagnetic energy of a given wavelength and emitting at a different wavelength. Examples include fluorescence, bioluminescence and phosphorescence. Luminescence can be caused by chemical or biochemical changes, electrical energy, subatomic motions, reactions in crystals, or other, generally non-thermal, stimulation of the electronic state of an atomic system. A “luminescent label” or “marker” is a molecular construction, is capable of emitting light, that is bound, either covalently, generally through a linker, or through ionic, van der Waals, hydrogen bonds, or any physical spatial constraint to another material, substance, or molecule. Preferably a luminescent label or marker is a molecule with aromaticity or a molecule with highly conjugated double bonds as typically found in fluorescent dyes, or quantum dots or combinations thereof.

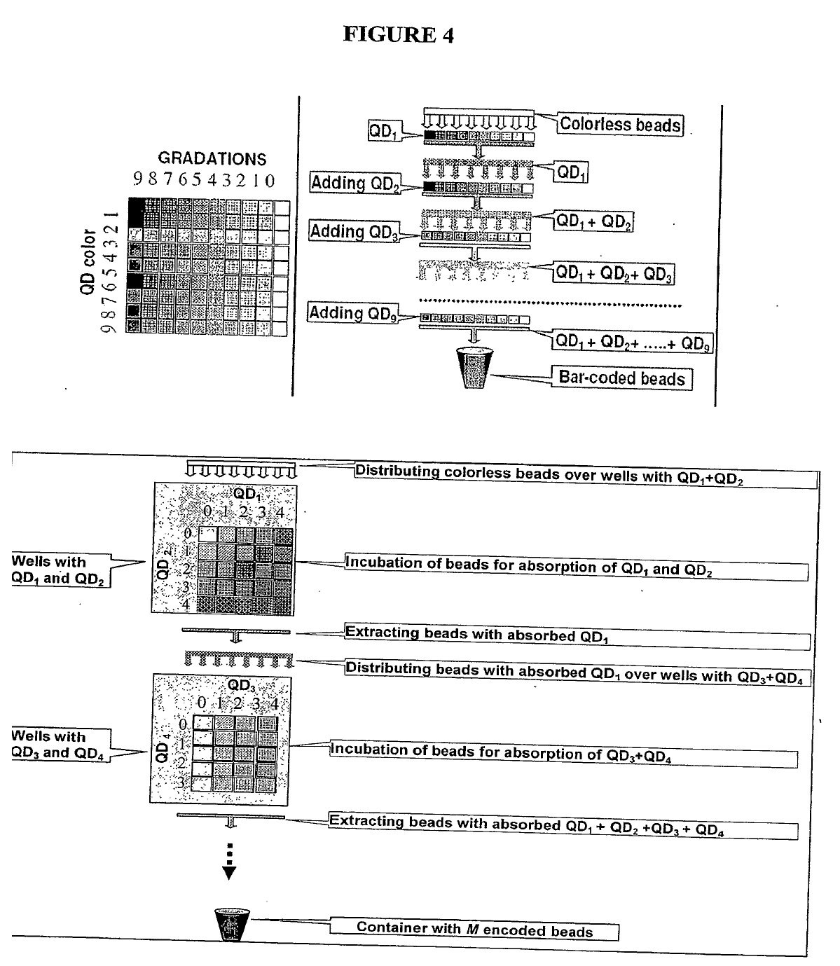

As used herein, “luminescent electromagnetic codes” or “spectral codes” mean the detectable collection of individually distinguishable wavelengths of electromagnetic radiation and corresponding distinguishable individual intensity that results from luminescence. In preferred embodiments the luminescent electromagnetic codes are in the visual region, i.e., gradations of visual colors. Example 2 describes creating beads with spectral codes and FIG. 4 illustrates creating the beads with more than three discrete visual colors. A “unique luminescent electromagnetic code” means a specific luminescent electromagnetic code.

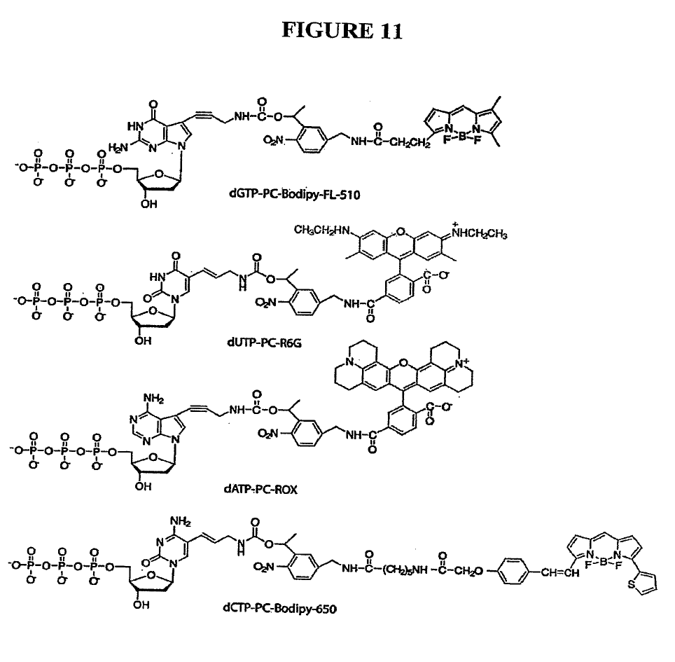

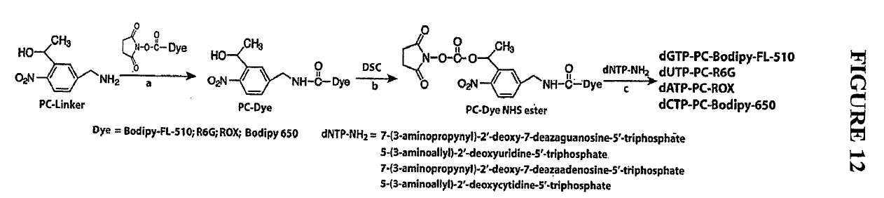

A “removable luminescent marker” is a luminescent marker that is detached upon exposure to a particular condition. An example of a removable luminescent marker attached to a nucleotide is provided in FIG. 11. Exemplary markers can be prepared (or as appropriately modified) as provided in Seo et al., Proc Natl Acad Sci USA. 2005; 102(17): 5926-5931 (FIG. 12). After these nucleotides are incorporated into a growing DNA strand in a solution-phase polymerase reaction, one may cleave the fluorophore using laser irradiation (≈355 nm).

As used herein, the term “ligate” in relation to nucleic acids and nucleotides means the process of joining two or more nucleic acids, nucleotides or combinations thereof by creating a covalent phosphodiester bond between the 3′ hydroxyl of one nucleotide and the 5′ phosphate of another. It is not intended to be limited to the actions of a DNA ligase, but also includes the actions of a DNA polymerase.

As used herein, the term “binding partners” refers to two molecules (e.g., proteins) that are capable of, or suspected of being capable of, physically interacting with each other such that the interaction changes a physical, chemical or biological property of one or both molecules acting independently. As used herein, the terms “first binding partner” and “second binding partner” refer to two molecular species that are capable of, or suspected of being capable of, physically interacting with each other. The terms “specific binding” and “specifically binding” when used in reference to the interaction between an antibody and an antigen describe an interaction that is dependent upon the presence of a particular structure (i.e., the antigenic determinant or epitope) on the antigen. In other words, the antibody recognizes and binds to a protein structure unique to the antigen, rather than binding to all proteins in general (i.e., non-specific binding).

As used herein, a “phenotype” means the observable physical or biochemical characteristics of an organism, such as, but not limited to, the onset of disease under environmental factors. The genetic makeup is believed to influence disease onsent. For example, a single-nucleotide polymorphism of the PTPN22 (protein tyrosine phosphatase, non-receptor type 22) gene, 1858C/T, has been found to be associated with many autoimmune diseases. A type 1 diabetes susceptibility is thought to be correlated to a locus on chromosome 10p11-q11 (provisionally designated IDDMIO); Sickle Cell Anemia is caused by a point mutation in the hemoglobin beta gene (HBB) found on chromosome 11p15.4; the APOE ε4 allele corresponds to susceptibility to late-onset Alzheimer's disease Saunders, A. M. et al. (1993) NeuroBiol. 43, 1467-72; the Factor V 1691G_A allele (FV Leiden) is involved in hereditary deep-vein thrombosis (Corder, E. H. et al. (1994) Nat. Genet. 7, 180-4) and several forms of the cytochrome p450 (CYP) gene affect drug metabolism (van der Weide, J. and Steijn, L. S. (1999) Ann. Clin. Biochem. 36, 722-9, Tanaka, E. (1999) Update: J. Clin. Pharm. Ther. 24, 323-9).

“Subject” means any animal, preferably a human patient, livestock, or domestic pet.

As used herein, the term “antibody” (or “antibodies”) refers to any immunoglobulin that binds specifically to an antigenic determinant, and specifically binds to proteins identical or structurally related to the antigenic determinant which stimulated their production. Thus, antibodies are useful in assays to detect the antigen that stimulated their production. Monoclonal antibodies are derived from a single clone of B lymphocytes (i.e., B cells), and are generally homogeneous in structure and antigen specificity. Polyclonal antibodies originate from many different clones of antibody-producing cells, and thus are heterogenous in their structure and epitope specificity, but they all recognize the same antigen. In some embodiments, monoclonal and polyclonal antibodies are used as crude preparations, while in preferred embodiments, these antibodies are purified. For example, in some embodiments, polyclonal antibodies contained in crude antiserum are used. Also, it is intended that the term “antibody” encompass any immunoglobulin (e.g., IgG, IgM, IgA, IgE, IgD, etc.) or fragment thereof, whether or not combined with another substituent by chemical linkage or as a recombinant fusion product, provided only that it is capable of serving as a binding partner with an antigen. Such antibodies may be obtained from any source (e.g., humans, rodents, non-human primates, lagomorphs, caprines, bovines, equines, ovines, etc.).

As used herein, the term “antigen” is used in reference to any substance that is capable of being recognized by an antibody. It is intended that this term encompass any antigen and “immunogen” (i.e., a substance which induces the formation of antibodies). Thus, in an immunogenic reaction, antibodies are produced in response to the presence of an antigen or portion of an antigen. The terms “antigen” and “immunogen” are used to refer to an individual macromolecule or to a homogeneous or heterogeneous population of antigenic macromolecules. It is intended that the terms antigen and immunogen encompass protein molecules or portions of protein molecules that contain one or more epitopes. In many cases, antigens are also immunogens, thus the term “antigen” is often used interchangeably with the term “immunogen.” In some preferred embodiments, immunogenic substances are used as antigens in assays to detect the presence of appropriate antibodies in the serum of an immunized animal.

The terms “antigenic determinant” and “epitope” as used herein to refer to that portion of an antigen that makes contact with a particular antibody variable region. When a protein or fragment (or portion) of a protein is used to immunize a host animal, numerous regions of the protein are likely to induce the production of antibodies that bind specifically to a particular region or three-dimensional structure on the protein (these regions and/or structures are referred to as “antigenic determinants”). In some settings, antigenic determinants compete with the intact antigen (i.e., the “immunogen” used to elicit the immune response) for binding to an antibody.

As used herein, the term “ELISA” refers to enzyme-linked immunosorbent assay (or EIA), Numerous ELISA methods and applications are known in the art, and are described in many references (See, e.g., Crowther, “Enzyme-Linked Immunosorbent Assay (BLISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). In addition, there are numerous commercially available ELISA test systems.

One of the ELISA methods used in the present invention is a “direct ELISA,” where an antigen in a sample is detected. In one embodiment of the direct ELISA, a sample containing an antigen is exposed to a supporting structure (e.g., a bead) under conditions such that antigen is immobilized on the structure in a manner that permits the antigen to be detected thereon directly using an enzyme-conjugated antibody specific for the antigen. Detected products of the reaction catalyzed by the enzyme indicates the presence of the immobilized antigen as well as the supporting structure to which it is bound.

In an alternative embodiment, an antibody specific for an antigen is detected in a sample. In this embodiment, a sample containing an antibody is exposed to a supporting structure (e.g., a bead) under conditions such that antibody is immobilized on the structure. The antigen-specific antibody is subsequently detected using purified antigen and an enzyme-conjugated antibody specific for the antigen.

In an alternative embodiment, an “indirect ELISA” is used. In one embodiment, an antigen (or antibody) is immobilized to a solid support (e.g., a bead) as in the direct ELISA, but is detected indirectly by first adding an antigen-specific antibody (or antigen), followed by the addition of a detection antibody specific for the antibody that specifically binds the antigen, also known as “species-specific” antibodies (e.g., a goat anti-rabbit antibody), available from various manufacturers known to those in the art (e.g., Santa Cruz Biotechnology; Zymed; and Pharmingen/Transduction Laboratories).

As used herein, the term “capture antibody” refers to an antibody that is used in a sandwich ELISA to bind (i.e., capture) an antigen in a sample prior to detection of the antigen. In one embodiment of the present invention, biotinylated capture antibodies are used in conjunction with avidin-coated solid support. Another antibody (i.e., the detection antibody) is then used to bind and detect the antigen-antibody complex, in effect forming a “sandwich” comprised of antibody-antigen-antibody (i.e., a sandwich ELISA).

As used herein, a “detection antibody” carries a means for visualization or quantitation, typically a conjugated enzyme moiety that yields a colored or fluorescent reaction product following the addition of a suitable substrate. Conjugated enzymes commonly used with detection antibodies in the ELISA include horseradish peroxidase, urease, alkaline phosphatase, glucoamylase and beta-galactosidase. In some embodiments, the detection antibody is an anti-species antibody. Alternatively, the detection antibody is prepared with a label such as biotin, a fluorescent marker, or a radioisotope, and is detected and/or quantitated using this label.

A “charge-coupled device” or “CCD” is an image sensor, consisting of an integrated circuit containing an array of linked, or coupled, light-sensitive capacitors. Preferably a photodiode converts light into an electronic signal for the unit.

A “dichroic mirror” is a color filter used to selectively reflect light of a range of colors while passing other colors.

As used herein, an “object” means a material thing.

As used herein, a “nucleotide” is a chemical compound that consists of a heterocyclic base, a sugar, and one or more phosphate groups. Preferably, the base nucleotide is a derivative of purine or pyrimidine, and the sugar is the pentose (five-carbon sugar) deoxyribose or ribose. Nucleotides are the monomers of nucleic acids, with three or more nucleotides bonded together forming a “nucleotide sequence.” A nucleic acid may be double-stranded or single-stranded. A “polynucleotide”, as used herein, is a nucleic acid containing a sequence that is greater than about 100 nucleotides in length. An “oligonucleotide”, as used herein, is a short polynucleotide or a portion of a polynucleotide. An oligonucleotide typically contains a sequence of about two to about one hundred bases. The word “oligo” is sometimes used in place of the word “oligonucleotide”.

Nucleic acid sequences are said to have a “5′-terminus” (5′ end) and a “3′-terminus” (3′ end) because nucleic acid phosphodiester linkages occur at the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus.

In certain embodiments, “unique sequence” of a nucleic acid are linked to a bead. This means that the nucleic acid sequence contains overlapping identical nucleotide bases. It is preferred, that the overlapping identical nucleotide bases correspond to a desired hybridization target sequence.

Hybridization means the coming together (annealling) of single-stranded nucleic acid with either another single-stranded nucleic acid or a nucleotide by hydrogen bonding of complementary base(s). Hybridization and the strength of hybridization (i.e., the strength of the association between nucleic acid strands) is impacted by many factors well known in the art including the degree of complementarity of the respective nucleotide sequences, stringency of the conditions such as the concentration of salts, the Tₘ (melting temperature) of the formed hybrid, the presence of other components (e.g., the presence or absence of polyethylene glycol), the molarity of the hybridizing strands and the G:C content of the nucleic acid strands.

As used herein, the term “primer” refers to an oligonucleotide, whether occurring naturally (e.g., as in a purified restriction digest) or produced synthetically, capable of acting as a point of initiation of nucleic acid synthesis when placed under conditions in which synthesis of a primer extension product complementary to a nucleic acid strand is induced (i.e., in the presence of nucleotides, an inducing agent such as DNA polymerase, and under suitable conditions of temperature and pH). The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and use of the method. It is also contemplated that primers can be used in PCR (see below) to artificially insert desired nucleotide sequences at the ends of nucleic acid sequences.

As used herein, the terms “complementary” or “complementarity” are used in reference to a sequence of nucleotides related by the base-pairing rules. For example, the sequence 5′ “A-G-T” 3′, is complementary to the sequence 3′ “T-C-A” 5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as for detection methods that depend upon hybridization of nucleic acids.

As used herein, the term “polymerase chain reaction” (“PCR”) refers to the method described in U.S. Pat. Nos. 4,683,195, 4,889,818, and 4,683,202, all of which are hereby incorporated by reference. These patents describe methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase (e.g., Taq). The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is a controllable parameter, determined by the relative positions of the primers with respect to each other. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (i.e., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are themselves efficient templates for subsequent PCR amplifications.

The term “isolated,” when used in relation to a nucleic acid, as in “isolated oligonucleotide” or “isolated polynucleotide,” refers to a nucleic acid that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs which encode a multitude of proteins. The isolated nucleic acid or oligonucleotide may be present in single-stranded or double-stranded form. When an isolated nucleic acid or oligonucleotide is to be utilized to express a protein, the oligonucleotide contains at a minimum the sense or coding strand (i.e., the oligonucleotide may single-stranded), but may contain both the sense and anti-sense strands (i.e., the oligonucleotide may be double-stranded).

### Nucleic Acid Sequencing Methods

Methods of DNA sequencing are generally described in Metzker, Genome Res., Dec. 1, 2005; 15(12): 1767-1776 and Shendure et al., Nature Reviews Genetics 5, 335-344 (2004). The Sanger-Sequencing method or chain termination or dideoxy method is a technique that uses an enzymatic procedure to synthesize DNA chains of varying length in four different reactions that contain diluted concentrations of individual dideoxy nucleotides mixed in with normal nucleotides. DNA replication is stopped at positions that are occupied by one of the four dideoxy nucleotide bases resulting in a distribution of nucleotide fragments since the normal nucleotides wilt properly incorporate. Unnatural ddNTP terminators replace the OH with an H at the 3′-position of the deoxyribose molecule and irreversibly terminate DNA polymerase activity. One determines the resulting fragment lengths to decipher the ultimate sequence. Electrophoretic separation of the deoxyribonucleotide triphosphate (dNTP) fragments is done with single-base resolution.

Regions that have proved to be difficult to sequence with conventional protocols can be made accessible through mutagenesis techniques. One can create devices that integrate DNA amplification, purification and sequencing by using microfabrication techniques. For example, a microfabricated circular wafer for 384-well capillary electrophoretic sequencing may be used. Reactions are injected at the perimeter and run towards the center, where a rotary confocal fluorescence scanner carries out the detection. In another example, single-stranded polynucleotides pass single-file through a hemolysin nanopore, and the presence of the polynucleotide in the nanopore is detected as a transient blockade of the baseline ionic current.

In sequencing by hybridization (SBH) differential hybridization oligonucleotide probes are used to decode a target DNA sequence. As described in Cutler, D. J. et al. ligh-throughput variation detection and genotyping using microarrays. Genome Res. 11, 1913-1925 (2001), to resequence a given base, four features are present on the microarray, each identical except for a different nucleotide at the query position (the central base of 25-bp oligonucleotides). Genotyping data at each base are obtained through the differential hybridization of genomic DNA to each set of four features.

In one example, DNA to be sequenced is immobilized on a substrate such as a bead, membrane or glass chip. One then carries out serial hybridizations with short probe oligonucleotides (for example, 7-bp oligonucleotides). Where specific probes bind the target DNA, they can be used to infer the unknown sequence. In another example, to re-sequence a given base, four features are present on the microarray, each identical except for a different nucleotide at the query position (the central base of 25-bp oligonucleotides). Genotyping data at each base are obtained through the differential hybridization of genomic DNA to each set of four features. Arrays of immobilized oligonucleotide probes are hybridized to a sample DNA. One provides oligonucleotide ‘feature’ per square unit, and each feature consists of multiple copies of a defined 25-bp oligonucleotide. For each base pair of a reference genome to be re-sequenced, there are four features on the bead. The middle base pair of these four features is an A, C, G or T. The sequence that surrounds the variable middle base is identical for all four features and matches the reference sequence. By hybridizing labeled sample DNA to the bead and determining which of the four features yields the strongest signal for each base pair in the reference sequence, a DNA sample can be rapidly re-sequenced. The data-collection method involves scanning the fluorescence emitted by labeled target DNA that is hybridized to an array of probe sequences.

Cyclic-array methods generally involve multiple cycles of enzymatic manipulation of an array of spatially separated oligonucleotide features. Each cycle queries one or a few bases, but thousands to billions of features are processed in parallel. Array features can be ordered or randomly dispersed. Cyclic sequencing methods that are non-electrophoretic are contemplated.

Pyrosequencing measures the release of inorganic pyrophosphate, which is proportionally converted into visible light by a series of enzymatic reactions. Unlike other sequencing approaches that use 3′-modified dNTPs to terminate DNA synthesis, the pyrosequencing assay manipulates DNA polymerase by single addition of dNTPs in limiting amounts. Upon addition of the complementary dNTP, DNA polymerase extends the primer and pauses when it encounters a noncomplementary base. DNA synthesis is reinitiated following the addition of the next complementary dNTP in the dispensing cycle. The light generated by the enzymatic cascade is recorded as a series of peaks called a pyrogram The order of the series corresponds to the order of complementary dNTPs incorporated and reveals the underlying DNA sequence.

A method called fluorescent in situ sequencing (FISSEQ) uses linkers containing either a disulfide bridge, which is efficiently cleaved with a reducing agent, or a photocleavable/degradable group with fluorescently labeled dNTPs. The cleavable linkers allow removal of the bulky fluorescent group following incorporation by DNA polymerase (FIG. 11).

In both FISSEQ and Pyrosequencing, progression through the sequencing reaction is externally controlled by the stepwise (that is, cyclical), polymerase-driven addition of a single type of nucleotide to an array of amplified, primed templates. Single nucleotide addition (SNA) methods such as pyrosequencing use limiting amounts of individual natural dNTPs to cause DNA synthesis to pause, which, unlike the Sanger method, can be resumed with the addition of natural nucleotides. Limiting the amount of a given dNTP is required to minimize misincorporation effects observed at higher concentrations.

In both cases, repeated cycles of nucleotide extension are used to progressively infer the sequence of individual array features (on the basis of patterns of extension/non-extension over the course of many cycles). Pyrosequencing may detect extension through the luciferase-based real-time monitoring of pyrophosphate release. In FISSEQ, extensions are detected off-line (not in real time) by using the fluorescent groups that are coupled to deoxynucleotides.

Another method of sequencing is based not on cycles of polymerase extension, but instead on cycles of restriction digestion and ligation. A mixture of adaptors including every possible overhang is annealed to a target sequence so that only the one having a perfectly complementary overhang is ligated. Each of the 256 adaptors has a unique label, Fₙ, which may be detected after ligation. The sequence of the template overhang is identified by adaptor label, which indicates the template overhang. The next cycle is initiated by cleaving with BbvI to expose the next four bases of the template.

After fluorescence activated cell sorting (FACS) (used to isolate beads instead of cells) isolates fluorescently labeled beads loaded with cDNAs, the cDNAs are cleaved with DpnII to expose a four-base overhang, which is then converted to a three-base overhang by a fill-in reaction. Fluorescently labeled (F) initiating adaptors containing BbvI recognition sites are ligated to the cDNAs in separate reactions, after which the beads are loaded into capillary array. cDNAs are then cleaved with BbvI and encoded adaptors are hybridized and ligated. Labeled decoder probes are separately hybridized to the decoder binding sites of encoded adaptors and, after each hybridization, an image of the bead array is taken for later analysis and identification of bases. The encoded adaptors are then treated with BbvI, which cleaves inside the cDNA to expose four new bases for the next cycle of ligation and cleavage. To collect signature data, a bead is tracked through successive cycles of ligation, probing, and cleavage by the fluorescent code.

In some embodiments the invention relates to isolated amplification. After amplification, a feature to be sequenced may contain thousands to millions of copies of an identical DNA molecule, although features might be spatially distinguishable. The amplification is done to achieve sufficient signal for detection.

Although the method for clonal amplification is generally independent of the method for cyclic sequencing, different routes may be used. In one method, amplification is done by simultaneously performing multiple picoliter-volume PCR reactions. In another example, one may use polony technology, in which PCR is performed in situ in an acrylamide gel. Because the acrylamide restricts the diffusion of the DNA, each single molecule included in the reaction produces a spatially distinct micron-scale colony of DNA (a polony), which can be independently sequenced. For massively parallel signature sequencing (MPSS), each single molecule of DNA in a library is labeled with a unique oligonucleotide tag. After PCR amplification of the library mixture, capture beads (with each bead bearing an oligonucleotide that is complementary to one of the unique oligonucleotide tags) is used to separate out identical PCR products.

Clonal amplification may be achieved using beads, emulsion, amplification, and magnetic properties. For example, an oil-aqueous emulsion parses a standard PCR reaction into millions of isolated micro-reactors, and magnetic beads are used to capture the clonally amplified products that are generated in individual compartments.

In some embodiments, the invention is related to the use of reversible terminators, i.e., nucleotides that terminate polymerase extension (for example, through modification of the 3′-hydroxyl group), but in a way that permits the termination to be chemically or enzymatically reversed. Cyclic reversible termination (CRT) uses reversible terminators containing a protecting group attached to the nucleotide that terminates DNA synthesis. For the reversible terminator, removal of the protecting group restores the natural nucleotide substrate, allowing subsequent addition of reversible terminating nucleotides. One example of a reversible terminator is a 3′-O-protected nucleotide, although protecting groups can be attached to other sites on the nucleotide as well. This step-wise base addition approach, which cycles between coupling and deprotection, mimics many of the steps of automated DNA synthesis of oligonucleotides. Reversible terminators provide for simultaneous use of all four dNTPs (labeled with different fluorophores).

In some embodiments, the invention relates to cyclic-array methods that attempt to dispense with the amplification step. Some methods comprise the extension of a primed DNA template by a polymerase with fluorescently labeled nucleotides. In other embodiments, deciphering homopolymeric sequences is accomplished by limiting each extension step to a single incorporation. Reversible terminators provide single-molecule detection with ample signal-to-noise ratio using standard optics for single-molecule detection. Sequence information can be obtained from single DNA molecules using serial single-base extensions and the use of fluorescence resonance energy transfer (FRET) to improve signal-to-noise ratio and the real-time detection of nucleotide-incorporation events through a nanofabricated zero-mode waveguide. By carrying out the reaction in a zero-mode waveguide, an effective observation volume in the order of 10 zeptoliters (10-21 liters) is created so that fluorescent nucleotides in the DNA-polymerase active site are detected.

In another embodiment, the invention relates to a single-molecule approach using nanopore sequencing. As DNA passes through a nanopore, different base pairs obstruct the pore to varying degrees, resulting in fluctuations in the electrical conductance of the pore. The pore conductance can be measured and used to infer the DNA sequence. Engineered DNA polymerases or fluorescent nucleotides provide real-time, base-specific signals while synthesizing DNA at its natural pace.

In some embodiments, the invention relates to replication of a single nucleic acid onto single magnetic beads, each containing thousands of copies of the sequence of the original DNA molecule. The number of variant DNA molecules in the population then can be assessed by staining the beads with fluorescent probes and counting them by using flow cytometry. Beads representing specific variants can be recovered through flow sorting and used for subsequent confirmation and experimentation.

Another method of sequencing employs engineered DNA polymerases labeled with a fluorophore such as Green Fluorescent Protein (GFP) and combined with an annealed oligonucleotide primer in a chamber of a microscope field of view capable of detecting individual molecules as provided in U.S. Pat. No. 6,982,146, hereby incorporated by reference. Four nucleotide triphosphates, each labeled on the base with a different fluorescent dye are introduced to the reaction. Light of a specific wavelength is used to excite the fluorophore on the polymerase, which in turn excites the neighboring fluorophore on the nucleotide by FRET. As nucleotides are added to the primer, their spectral emissions provide sequence information of the DNA molecule.

### Quantum Dots

Quantum dots are semiconductor particles preferably with diameters of the order of 2-10 nanometers, or roughly 200-10,000 atoms. Their semiconducting nature and their size-confinement properties are useful for optoelectronic devices and biological detection. Bulk semiconductors are characterized by a composition-dependent bandgap energy, which is the minimum energy required to excite an electron to an energy level above its ground state, commonly through the absorption of a photon of energy greater than the bandgap energy. Relaxation of the excited electron back to its ground state may be accompanied by photon emission. Because the bandgap energy is dependent on the particle size, the optical characteristics of a quantum dot can be tuned by adjusting its size.

A wide variety of synthetic methods for making quantum dots are known, including preparation in aqueous solution at room temperature, synthesis at elevated temperature and pressure in an autoclave, and vapor-phase deposition on a solid substrate. Alivisatos, Science 271:933-937, 1996 and Crouch et al., Philos. Trans. R. Soc. Lond., Ser. A. 361:297-310, 2003. Most syntheses yielding colloidal suspensions of quantum dots involve the introduction of semiconductor precursors under conditions that thermodynamically favor crystal growth, in the presence of semiconductor-binding agents, which function to kinetically control crystal growth to maintain their size within the nanoscale.

Because the size-dependent properties of quantum dots are most pronounced when the nanoparticles are monodispersed, it is preferable to produce quantum dots with narrow size distributions. A synthetic method for monodisperse quantum dots (<5% root-mean-square in diameter) made from cadmium sulfide (CdS), cadmium selenide (CdSe), or cadmium telluride (CdTe) is described in Murray et al., J. Am. Chem. Soc. 115:8706-8715, 1993. Generating quantum dots that can span the visible spectrum are known, and CdSe has become the preferred chemical composition for quantum dot synthesis. Many techniques are possible for post-synthetically modified quantum dots, such as coating with a protective inorganic shell, Dabbousi, et al., J. Phys. Chem. B 101:9463-9475, 1997 and Hines & Guyot-Sionnest, J. Phys. Chem. 100:468-471, 1996, surface modification to render colloidal stability Gerion, et al., J. Phys. Chem. B 105:8861-8871, 2001 and Gao et al., J. Am. Chem. Soc. 125:3901-3909, 2003, and direct linkage to biologically active molecules. Bruchez et al., Science 281:2013-2016, 1998 and Chan & Nie, Science 281:2016-2018, 1998.

A preferred scheme of synthesis involves four steps: (1) synthesis of the quantum dot core, most often CdSe, in a high-temperature organic solvent; (2) growth of an inorganic shell (usually zinc sulfide, ZnS) epitaxially on the core to protect the optical properties of the quantum dot; (3) phase transfer of the quantum dot from organic liquid phase to aqueous solution; and (4) linkage of biologically active molecules to the quantum dot surface to render functionality, or linkage of biologically inert polymers to minimize biological activity.

One synthesis procedure for monodisperse quantum dots involves the addition of semiconductor precursors to a liquid coordinating solvent at high temperature. The coordinating solvent preferably consists of trioctylphosphine oxide (TOPO) and trioctylphosphine (TOP), which contain basic functional groups that can bond to the quantum dot surface during growth to prevent the formation of bulk semiconductors. The alkyl chains from coordinating ligands extend away from the quantum dot surface, rendering the quantum dots sterically stable as colloids, dispersible in many nonpolar solvents. In a preferred synthesis of CdSe, room-temperature quantum dot precursors, dimethylcadmium and elemental selenium dissolved in liquid TOP, are swiftly injected into hot (290-350° C.) TOPO, immediately initiating nucleation of quantum dot crystals. CdSe nucleation and growth is favored thermodynamically, because the precursors are introduced at concentrations well above the solubility of the resulting semiconductor. However, crystal growth is kinetically controlled by monomer diffusion, due to the high viscosity of the solvent, and is also controlled through the reaction rate of monomers at the quantum dot surface, due to strong binding of the coordinating solvent with semiconductor precursors and quantum dot surfaces. A high temperature at injection overcomes the steric/kinetic barrier, allowing precursor association and nucleation. The swift drop in temperature, combined with the drop in monomer concentration (due to the nucleation of many small quantum dot crystals), stops nucleation within seconds after injection, allowing even and homogeneous growth on similarly sized nuclei. This separation of nucleation and growth is responsible for the monodispersity of the final quantum dots. Also, the use of a hot solvent yields semiconductor nanoparticles that are highly crystalline, while minimizing thermodynamically unfavorable lattice defects.

At the focusing point, it is desirable to quench the reaction, usually by decreasing the temperature until crystal growth is negligible. This synthesis procedure is preferable for quantum dots composed of CdSe, although quantum dots with other compositions may be synthesized in coordinating solvents. Variations on this synthesis use alternate molecular precursors for CdSe, including but not limited to cadmium oxide, dimethylcadmium, and cadmium acetate, combined with TOP-Se, various coordinating ligands including but not limited to alkylamines and alkanoic acids and the coordinating solvent may be replaced with a noncoordinating solvent, like octadecene, containing a small amount of coordinating ligand. CdSe quantum dots with diameters between 2 and 8 nm, have emission wavelengths from (450-650 nm) spanning the entire visible spectrum. By also adjusting the quantum dot composition (ZnS, CdS, CdSe, CdTe, PbS, PbSe, and their alloys), it is possible to span the wavelength range 400-4000 mm. Adjusting the solvent characteristics and initial precursor concentration further results in manocrystals with diverse shapes, like rods and tetrapods.

When designing quantum dot cores for a specific wavelength region, the first choice is to select a chemical composition, since quantum dots are preferred for a certain wavelength range for each composition. For example, CdSe quantum dots may be tuned to emit between 450 and 650 nm, while CdTe quantum dots may be tuned to emit from 500 to 750 nm. The quantum dot diameter is then chosen to determine the specific wavelength of emission, and the quantum dots are then generated through focused particle growth using the synthesis parameters. The resulting quantum dots are coated in coordinating ligands and suspended in a crude mixture of the coordinating solvent and molecular precursors. Most quantum dots are highly hydrophobic, and can be isolated and purified from the reaction mixture, either through liquid-liquid extraction (a mixture of hexane and methanol), or through precipitation from a polar solvent (methanol or acetone) that dissolves the reactants and coordinating ligands, but not the quantum dots. The pure core quantum dots are then used as substrates for further modification.

Because quantum dots have high surface area to volume ratios, a large fraction of the constituent atoms are exposed to the surface, and therefore have atomic or molecular orbitals that are not completely bonded. These “dangling” orbitals may form bonds with organic ligands such as TOPO. This leads to an electrically insulating monolayer that serves to “passivate” the quantum dot surface by maintaining the internal lattice structure and protecting the inorganic surface from external effects. However, the bond strength between the organic ligand and the semiconductor surface atom is typically much lower than the internal bond strength of the semiconductor lattice, and desorption of ligands makes the core physically accessible. For this reason, it is preferred to grow a shell of another semiconductor on the QD surface after synthesis. By using a shell of wider bandgap than the underlying core, strong electronic insulation results in enhanced photoluminescence efficiency, and a stable shell provides a physical barrier to degradation or oxidation. As an example, to passivate CdSe quantum dots with ZnS, the cores are purified to remove unreacted cadmium or selenium precursors, and then resuspended in a coordinating solvent. Molecular precursors of the shell, usually diethylzinc and hexamethyldisilathiane dissolved in TOP, are then slowly added at elevated temperatures. The temperature for growth of ZnS on CdSe is chosen such that it is high enough to favor epitaxial crystalline growth, but low enough to prevent nucleation of ZnS crystals and Ostwald ripening of CdSe cores. Normally, this is a temperature around 160-220° C. The (core)shell (CdSe)ZnS nanocrystals may then be purified just like the cores. Although having a shell is preferred, in certain embodiments uncapped CdSe cores are used.

Quantum dot syntheses may be performed directly in aqueous solution generating quantum dots ready to use in biological environments. Two strategies that may be used to make hydrophobic quantum dots soluble in aqueous solution include, but are not limited to, ligand exchange, and coating with an amphiphilic polymer. For ligand exchange, a suspension of TOPO-coated quantum dots are mixed with a solution containing an excess of a heterobifunctional ligand, which has one functional group that binds to the quantum dot surface, and another functional group that is hydrophilic. Thereby, hydrophobic TOPO ligands are displaced from the QD through mass action, as the new bifunctional ligand adsorbs to render water solubility. Using this method, (CdSe)ZnS QDs may be coated with mercaptoacetic acid and (3-mercaptopropyl) trimethoxysilane, both of which contain basic thiol groups to bind to the quantum dot surface atoms, yielding quantum dots displaying carboxylic acids or silane monomers. These methods generate quantum dots that are useful for biological assays. More preferably, one may retain the native TOPO molecules on the surface, and covert the hydrophobic quantum dots with amphiphilic polymers. These methods yield quantum dots that can be dispersed in aqueous solution and remain stable for long periods of time due to a protective hydrophobic bilayer encapsulating each quantum dot through hydrophobic interactions. It is preferable that the quantum dots are purified from residual ligands and excess amphiphiles before use in biological assays by ultracentrifugation, dialysis, or filtration.

In preferred water solubilization methods, quantum dots are often covered with carboxylic acid groups, and the quantum dots are negatively charged in neutral or basic buffers. Preferred schemes used to prepare quantum dot bioconjugates rely on covalent bond formation between carboxylic acids and biomolecules. Since the QD surface has a net negative charge, positively charged molecules can also be used for electrostatic binding, a technique that may be used to coat quantum dots with cationic avidin proteins and recombinant maltose-binding proteins fused with positively charged peptides. Alternatively, biomolecules containing basic functional groups, such as amines or thiols, may interact directly with the quantum dot surface as ligands. If biomolecules do not innately contain groups for direct quantum dot binding, they may be modified to add this functionality. For example, nucleic acids and peptides may be modified to add thiol groups for binding to quantum dots. Surface modification has also become modular through high-affinity streptavidin-biotin binding. Quantum dot-streptavidin conjugates are convenient for indirect binding to a broad range of biotinylated biomolecules. Quantum dots can be coated with inert hydrophilic polymers, such as polyethylene glycol (PEG), which act to reduce nonspecific adsorption and to increase colloidal stability. Biocompatible quantum dots are may be conjugated to a variety of functional biological molecules, like streptavidin, biotin, or monoclonal antibodies.

Multiple quantum dots may be precisely doped in mesoporous silica beads. For example, quantum dots may be coated with a layer of tri-n-octylphosphine oxide (TOPO). Mesoporous materials may be synthesized by using pore generating templates such as self-assembled surfactants or polymers. Preferably mesoporous silica beads (5 μm diameter) with pore sizes of 10 or 32 nm are coated with a monolayer of Si—C₁₈H₃₇ (octadecyl, an 18-carbon linear-chain hydrocarbon).

Single-color doping may be accomplished by mixing porous beads with a controlled amount of quantum dots in an organic solvent such as butanol. For example, 0.5 mL of a 4-nM quantum dot solution (chloroform) may be mixed with one million porous beads in 2-5 mL of butanol, yielding a doping level of 1.2 million dots per bead. For the 10-nm pore beads, more extended times may be used. For multicolor doping, different-colored quantum dots may be premixed in precisely controlled ratios. Porous beads may be added to an aliquot of this premix solution. Doped beads may be isolated by centrifugation and washed three times with ethanol.

Quantum dots may be clustered together. Typically these clusters are coated with an additional shell, e.g., zinc sulfide. These clusters can be coated with a polymer. Chemical modification of the polymer allows the surface of the nanocrystals to be modified such that biomaterials and molecules can be attached to a polymer coat. For example, polystyrene can be used to coat a nanocrystal. The polystyrene can be hydroxylated to form phenol groups. Reaction of the phenol groups with p-hydroxybenzyl bromide results in substitution of the bromide to provide a hydroxybenzyl surface. The benzyl hydroxyl group can be converted to an alkyl halide or amine as desired and coupled to an amino acid as typically utilized in resin mediated amino acid solid phase synthesis. An amino acid sequence on the exterior of the bead can be the epitope of an antibody that may or may not itself be fluorescently labeled. In a similar fashion, a nucleic acid sequence may be conjugated to the polymer surface. A conjugated nucleic acid sequence on the exterior of the bead can hybridized to a complimentary sequence that may or may not contain an additional fluorophore.

### Telomerase

Telomerase is a ribonucleoprotein that maintains chromosomal telomere length. Telomerase is not active in nonmalignant somatic cells, but is activated in most human cancers. Telomerase activity may be used as a cancer marker, especially when used in conjunction with conventional cytology. Functional telomerase is present in about 90% of all human cancers but is generally absent from benign tumors and normal somatic (except germ line and stem) cells. The detection of telomerase activity has the highest combination of sensitivity (60-90%) and clinical specificity (94-100%) when compared to other screening methods for identifying cancers.

The ends of chromosomes consist of thousands of double-stranded (ds) TTAGGG repeats called telomeres that have several functions. In normal somatic cells, telomere length is progressively shortened with each cell division, eventually leading to cell death. In contrast, unlimited proliferation of most immortal and cancer cells is highly dependent on the activity of telomerase, which compensates for replicate telomere losses by elongating the existing telomere with TTAGGG repeats, using its own RNA component as a template.

The detection of telomerase activity may be based on the telomeric repeat amplification protocol (TRAP), which employs the ability of telomerase to recognize and elongate, in vitro, an artificial oligonucleotide substrate, TS, and then uses PCR to amplify the extended DNA products. Real-time quantitative TRAP (RTQ-TRAP) combines the conventional TRAP assay and a real-time PCR based on SYBR Green. A more specific telomerase detection was demonstrated using TRAPeze XL™ kit that employs Amplifluor™ primers.

One factor limiting sensitivity of real-time PCR is high background fluorescence of probes that are not bound to the PCR product. Separating DNA fragments with capillary electrophoresis (CE) and detecting their laser-induced fluorescence (LIF) eliminates the background fluorescence, allows concentration of the PCR product due to the sample stacking during the electrokinetic injection, and thus improves sensitivity. To further lower the detection threshold, one may combined CE-LIF with a single photon detector (SPD). Sensitivity of SPDs is intrinsically very high due to their high quantum yield and extremely low dark count. In addition, the electric output of an SPD can be directly processed by digital circuitry. Hence, signal amplification, recording and processing steps do not add any noise to the detected signal as opposed to common LIF systems.

### Document Authenticity

At times it is desirable to identify copies of printed materials that look identical to the original, such as bank securities, manuscripts, identity cards, checks, cash. In some embodiments the invention relates to the use of one or more security codes or marks embedded in a document as deterrents to theft or counterfeiting. These codes may appear as watermarks, holograms, fluorescent dyes in ink, bar codes, or number codes.

As used herein a “document” means something that can be used to furnish evidence or information. Preferably the document is a written or printed paper that bears the original, official, or legal form; however, it is not intended to be limited thereto. There are many types of identity documents that generally consist of a picture, name, address, fingerprints, number code, etc. Examples would include national identity cards, passports, driver's licenses, and company ID cards/keys. Other examples of documents include bank securities and cash.

In some embodiments, the invention relates to the use of a luminescent signature of the beads used in the dye to determine the authenticity of a document. For example, an ink may contain a set of beads that contain differing concentrations of quantum dots. The ink may be applied to a document. Detection of the differing quantum dots by exposure to ultraviolet light provided different color and there relative concentrations in provides a different intensity of color; thus a luminescent signature or spectral is created depending on the beads and the quantum dots used.

In some embodiments, beads with spectral codes are selected and applied to a document either during the printing process or during post production, (or to a spot gloss coat, or the plastic laminate used to physically protect critical documents), the document can be readily identified as authentic in as little as a few seconds using fluorescence reader. The composition of the codes remains secure, since only a key identifier appears on the screen. To further enhance the covert security of the technique, multiple invisible tags can be added internally to the document or to the back surface of the paper or document, and/or its packaging through the inks used in the printing process or embedded in the substrate itself.

Infrared visible inks may be readable or disappearing. When printed they can look the same but when viewed under infrared light, one will be readable and one will disappear. One example of using these two inks as a security feature would be to print a bar code using both inks. Print the actual readable area of the bar code with the infrared readable ink and other areas of the bar code with the infrared disappearing ink but making it look like a regular bar code. When read by a bar code scanner, only the infrared readable is read by the scanner. If a forger tries to duplicate the bar code as it looks on the printed document, using regular inks, the bar code would be rejected when read by the scanner because the scanner would read the entire bar code. Visible infrared ink is available for wet or dry offset printing.

Photochromic ink can be colored or colorless. When it is exposed to UV light it instantly changes colors. Once the source of UV light is removed it will change back to its original color. The unique properties of photochromic ink cannot be reproduced by a scanner or copier. The authenticity of a document with photochromic ink on it can be checked by exposure to sunlight, UV lights or other strong artificial lights. This ink may be wet or dry offset with flexographic printing.

### EXAMPLES

### Example 1 Detection of Beads in Capillary Flow

Streptavidin-coated polystyrene 6 μm beads were labeled with fluorescein by incubation with biotinylated antibody followed by a corresponding fluorescence conjugated detection antibody. In order to obtain a vehicle solution for carrying beads without sedimentation, Applied Biosystems Polymer (Performance Optimized Polymer-4) was premixed with PBS at a 1:1 ratio. A pump was used to push beads at a constant speed through a 25 cm long, 51 μm ID capillary.

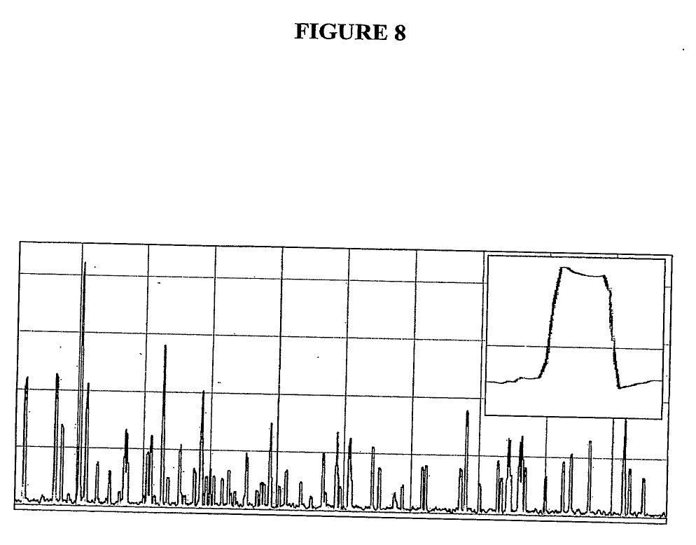

Fluorescence was excited at 488 mm with a 4 mW Ar-ion laser. Each peak of fluorescence corresponds to a single 6 μm bead passing the 60 mm cross-section of the laser beam (see FIG. 8). Minimum peak amplitude detected in this series was about 104 counts/second at a background level of about 20,000 counts/sec with signal-to-noise ration of higher than 3. Experiments with unlabeled beads showed that they produced no signal above the background level.

### Example 2 Spectral Encoding of Micro-Beads (FIG. 4)

Assume one has D types of luminescent dyes with different luminescent spectra diluter in a buffer and having G₁ gradations in each of said dye type (1≦i≦D). Assume that ones want to create a set of N beads which carry C color codes, where

$$C \leq {\prod }_{i = 1}^{D} {G}_{i} .$$

In order to obtain N beads encoded with said codes one does the following: Create W well plates, each well plate comprises wₖ wells so that

$${\prod }_{k = 1}^{W} {w}_{k} = C , \\ where$$

$${w}_{k} = {\prod }_{i = 1}^{{d}_{j}} {G}_{i}$$

and dⱼ is a number of types of luminescence dyes such that

$${\sum }_{j = 1}^{W} {d}_{j} = D .$$

a) Take the first set of d₁ luminescent dyes, prepare w₁ different combinations of the first set of said luminescent dyes and place them into w₁ wells of the first well plate, one dye combination per one well; Repeat the same procedure W times, every time choosing different sets of dⱼ dyes and filling different well tray;
b) Distribute M colorless porous beads between said w₁ wells of the first well plate;
c) Incubate said M beads in said well plate so that said luminescent markers are absorbed by said beads (beads' doping procedure);
d) Extract said M beads from said well plate and separate said M beads from said d₁ luminescent dyes;
e) Mix said extracted M beads together;
f) Distribute said M beads between w₂ wells of the second well plate;
g) Repeat steps d)-g) W-2 times;
h) Repeat steps d)-f) one more time;
i) Place said M spectrally encoded beads into a container

After the procedure of the beads' doping is completed, one obtains a solution which contains C distinct codes, called bead families. To each bead family one assigns a label from Ito C. Each family consists of many members—the beads which carry the same code. If during the encoding procedure a very large number of beads M was always evenly distributed between reaction wells, each family will consists of approximately K members where K=M/C±\sqrt{M/C}. Let us call L beads which are randomly taken from the mix of beads after the encoding procedure a set of beads and let us estimate a number of unique codes in the set for L≈M/K≈C. Suppose that one has C pits, likewise labeled by a number from 1 to C. When one selects a single bead with a label m from the global reservoir of M beads, one places this bead into the pit with the same label m. Given L randomly selected beads, one wishes to know how many pits contain 1 and only 1 bead. In order to derive the probability p(C,L) of obtaining 1 and only 1 bead in a given pit after placing L randomly selected beads, one computes the probability of each possible way in which one may succeed and summed these probabilities. For large M and C one obtains [symbol] C,L)=L/N exp (−L/C). Maximum value for p is ˜0.36 and it is obtained when L=C. One posits that the problem is equivalent to the Bemoulli problem, with the number of trials C, the success probability p, the mean number of successes N_{SUCCESS}=C×p distributed with the standard deviation σ=\sqrt{Cp(1−p)}. Thus, by taking a fraction 1/K from the mix containing M beads one obtains a set of L=M/F=C±\sqrt{C} beads containing ˜36% of uniquely coded beads.

To create 10⁹ distinct spectral codes in quantum dots, one uses 9 distinct colors with 10 gradations of intensity. Since intensity of fluorescence produced by a bead doped with quantum dots is proportional to the number of quantum dots embedded in the bead, one dopes beads with different amounts of quantum dots in order to create intensity gradations. The distance between average intensities of the neighboring gradation is double the standard deviation.

A large amount of colorless porous beads are distributed between ten wells filled with solutions of different concentrations of quantum dot with a first color (QD₁ see FIG. 4). After embedding QD₁ into the beads, contents of all 10 wells are mixed together. The beads are washed and evenly and randomly distributed between the next set of ten wells filled with different concentrations of QD₂. The procedure is repeated for each distinct color.

One encodes porous polystyrene beads with quantum dots as described in Gao & Nie Analytical Chemistry 2004, 76, 2406-2410. One uses ZnS-capped CdSe core shell quantum dots coated with a layer of TOPO. One uses polystyrene porous micro-beads with pores between 10 and 30 nm. One does single color quantum dot doping by injecting a controlled amount of quantum dots into porous beads suspended in butanol. The mixture is stirred until essentially only quantum dots are left in the supernatant solution. One isolates the beads by centrifugation and washes them with ethanol.

It is preferred to extract and wash the beads in a manner that the beads continue to be exposed to a liquid environment in order to prevent gaseous bubbles forming within the porous beads hindering absorption of the quantum dots. In one embodiment, this is accomplished by diluting the suspension, allowing the beads to settle in the bottom of the well, removing a portion of the solution such as, by sucking out the top half of the solution with a pipette. If the well contains a permeable membrane such as a glass frit, it is possible to apply a positive pressure to the membrane to keep the solution in the well during the doping process and then remove a portion of the solution by applying suction.

In addition, in order to reduce the number of extracting, washing, and transferring steps it is preferable to create wells with more than one color quantum dot in each well. For example, it is contemplated that one can utilized a plate with 96 wells and put varying concentrations of quantum dot having a red color in rows and varying concentrations of quantum dots having a green color in the columns. Thus, after the beads absorb the quantum dots, they have two color indicators. It is also contemplated that more than two colors can be used. For example, the multiple plates described above can have varying concentrations of a third, fourth, fifth, ect, color indicators in each plate. Having more wells with more colors variations allows the production of more beads with unique color concentration before extracting, washing, recombination and redistribution. After that, a second, third, fourth, ect, doping is done providing further diversity.

In preferred embodiments, it is also contemplated that it may be desirable to utilize different relative concentrations of quantum dot colors in the beads depending on the application. As described, in some embodiments of the invention, detection of the fluorescence of each bead is accomplished using a set of mirrors that deflect or pass determined wavelengths of electromagnetic radiation. Light intensity is lost each time light reflects or passes through a mirror. Thus, depending on the position of the color detector, such as a CCD detector, in the system, it may be desirable to increase the relative concentration of quantum dots having a color where the detection instrument is placed at the in a location that requires the fluorescence to pass through the most mirrors. For example, in some embodiments, it is preferred to increase the concentration of green fluorescing quantum dots in the bead concentration and position the instrument for detection of the green color in a location having the most number of mirrors, and it is preferred to decrease the concentration of the red fluorescing quantum dots in the bead concentration and position the instrument for detection of the red color in a location having the least number of mirrors.

### Example 3 Spectral Identification of Spectrally-Coded Beads with Labeled DNA Fragments

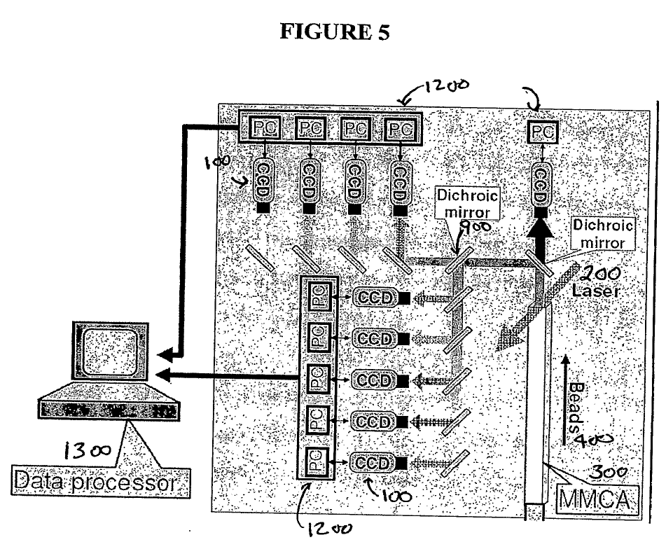

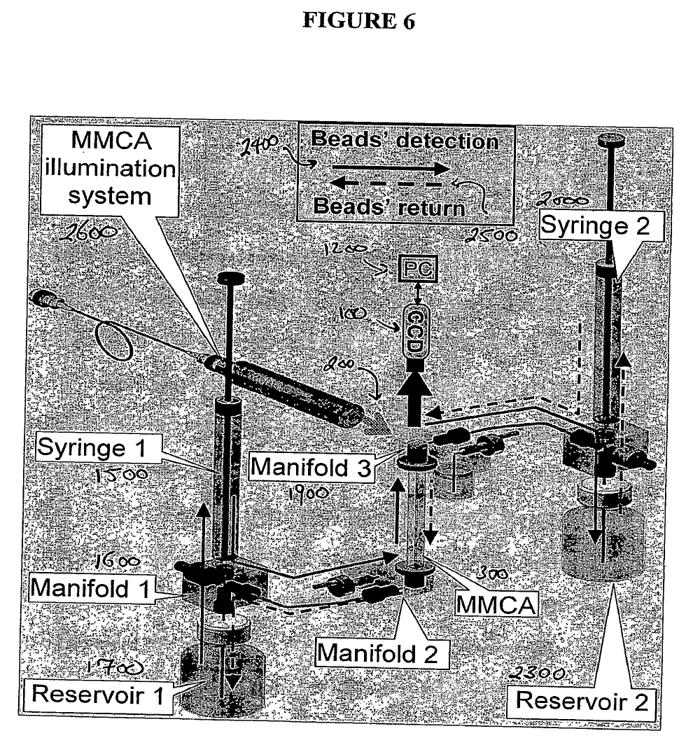

The system comprises an optical detection subsystem, a fluidic subsystem, and a data acquisition subsystem (see FIGS. 5 and 6). The optical system comprises an Ar-ion laser (488 nm, 0.5-1 W), optical line generator(s) (to illuminate the entire cross section of the monolith micro-capillary array), array lens to transfer the fluorescent image outside of the monolith micro-capillary array's manifold, low-pass 5 O.D. filter for rejection of the laser wavelength, a set of dichroic mirrors (90% transparency/90% reflection) and a set of CCD cameras (Cascade 128+, from Photometrics) with narrow band-pass filters to minimize spectral cross-talk between different quantum dot types. One uses near-infrared dyes for DNA labeling. One detects fluorescence from labeled DNA by the CCD camera. For 5 μm beads, one may alternatively use a CMOS camera (NW D1024-160 from Photonfocus AG).

One dilutes a set of beads coded with the quantum dot colors in a buffer and pushes them through the capillary channel. The top of the channel is illuminated by a laser beam at 488 nm. When the beads approach the top of the channel and cross the laser beam, the fluorescence excited in the beads passes through the laser rejection filter, the relay lens, and the system of dichroic mirrors. Fluorescent images are detected by the CCD cameras. An additional CCD camera on top of the column detects fluorescence from the labeled DNA (see first dichroic mirror in FIG. 5 near laser). All CCD cameras have dedicated single board computers, CCD computers, which transfer a synchro-signal to the CCD cameras so that frames in all CCD cameras are synchronized and all colors emitted by a bead are detected simultaneously. Color images are recorded and the acquired data is transferred to a computer processor for further processing analysis and storage.

Data Acquisition

Each CCD camera has an individual single board computer connected to it. Initial data acquisition and processing will happen on these computers. The initial data processing includes image processing and yields a file that contains fluorescence intensities measured from each capillary of the array for each frame. The acquired data is transferred from the CCD computers to a computer PROCESSOR through a network.

Disambiguation of Beads

One has a machine capable of reading all colors on a single bead. For the purposes of this discussion, one assumes that K is the number of different colors on this bead, and G is the number of gradations per color. Thus, one has G^{K} possible distinct beads.

When one measures a bead, one get thusly a K-dimensional vector V, with values 0-G for each element. As one cannot measure the intensity of each color in absolute terms, but one can measure their relative intensity. One normalizes the vector to the range 0-G, via the following algorithm wherein V[i] denotes the i-th element of vector V, max(V) denotes the maximum distinct element of V);

maximum=max(V)
for i=1 to K:

V[i]=V[i]/maximum

After this procedure, one has a normalized record of each bead. Having recorded a large number of them, one counts the number each V occurs in the set of beads.

This can be accomplished efficiently with the following algorithm:

One represent the set of beads of size N as a trie (a prefix tree), where each node of the prefix tree is a gradation. Once a terminal node of the trie is reached, its count is incremented by 1. To count the number of color combinations that occur only once, one traverses the trie, and returns only the leaf nodes with the count of 1.

The prefix tree has a depth G, and the number of nodes N. It is clear that insertion into the prefix tree take O(G) time. Thus, inserting N elements takes O(N*G) time. In practical cases, G is quite small, and thus, the total running time of the insertion operation is effectively O(N), which is optimal, as this is the size of the input.

A prefix tree is an ordered tree data structure that is used to store an associative array where the keys are ordered lists (vectors). Unlike a binary search tree, no node in the tree stores the key associated with that node; instead, its position in the tree shows what key it is associated with. All the descendants of any one node have a common prefix of the string associated with that node, and the root is associated with the empty string. Unfortunately, a trie is fundamentally a random-access data structure, and has to be kept in main memory. For 1 billion beads, this would require over 10 gigabytes of memory for the trie. One solution is to somehow cluster the read beads into chunks that fit into main memory, and yet, for a particular vector, always include all occurrences of it. On may do this in the following fashion:

Prior to inserting the read vectors into the trie, one performs bucketization based on the hashing on each vector, and mapping the result into the integer range 0-N/(memory size), resulting in roughly N/(memory size) chunks. Thus, hash(V) mod (N/(memory size)) gives one the bucket in which the current vector should reside. One appends V to the bucket in memory, and once it grows above a predefined size (1 MB, for example), one appends it to the bucket-file on disk, and one empties the memory copy. The procedure takes O(N) time, as a hash takes O(1) time to compute.

A property of the hash functions is that if two hashes are the same, then the two inputs are the same. Thus, if one stores a vector V in bucket B1, then all vectors which are the same as V were stored in the bucket B1. So, the algorithm looks like:

for each V in input:

append V to Bucket number ([hash(V) mod (N/(memory size)))
if Bucket number ([hash(V) mod (N/(memory size))) is full:

save it on disk
empty it

for each B in bucket:

create trie
for each V in B:

insert V into trie
increment counter on the leaf node of V in the trie by 1

traverse trie, and print out all leafs with counter = 1

This algorithm takes O(N)+O(G×N) time, and, for expected datasets, entirely fits into main memory. The time taken is dominated by the reading and writing of buckets onto the disk. As stated, the access pattern of the algorithm is sequential, thus, one can estimate the possible throughput by dividing the expected amount of data by the throughput rate of the disk. For a modern computer, a 100 MB/sec throughput rate is note unreasonable. Thus, a dataset with N=1×10⁹ will take 1×10¹⁰ bytes, which is 10 GB. As one need 4 s passes (1 to read in the data, 1 to store the buckets, 1 to read the buckets for insertion into the trie, and 1 to store the results) one ends up writing 40 GB, which, at 100 MB/s throughput, takes 400 seconds, or a bit under 10 minutes.

The automated fluidic system allows multiple readings of the same bead set, after the fashion of data acquisition in the DNA sequencing system, where the same set of beads is detected after each extension cycle. The system consists of two syringes, 4 manifolds, monolith multi-capillary array, valves, motors, and actuators. One loads the set of beads into the first syringe and pushes through the manifolds and array for each frame. The acquired data is transferred from the CCD computers to a computer processor through a network (see FIG. 6).

The detection rate can be determined as follows: r_{DET}=(N_{CAP}×f_{CCD})/l where f_{CCD} is CCD frame rate and l is a number of frames needed for detection of 1 bead. For example, for l=5 and f_{CCD}=500/s and 1,000≦N_{CAP}≧100,000 we will have 1051/s≦r_{DET}≧1071/s.

The optical system includes multiple elements that introduce loss of fluorescence (FIG. 5). The total loss includes: light collection loss (we assume 2% total efficiency with both relay lens and CCD objectives); mirror loss (maximum mirror loss is estimated as 0.59 for 5 mirrors); detection efficiency (minimum 40% for selected CCD cameras) Therefore the total efficiency of the optical system will be 0.5%. Dichroic mirrors positioned in a row will cause uneven fluorescent signals depending on the mirror position. If mirrors have identical loss η the signal detected from i-th mirror will be ηⁱ. Therefore, if we increase the amount of quantum dots N_{QD} detected through this mirror by 1/ηⁱ we will obtain the same fluorescence intensity for all mirrors.

The estimated fluorescent signal which can be obtained from the porous bead of diameter d can be calculated assuming that the intensity of the signal is directly proportional to the number of quantum dots in the bead. The described above process of the sequential addition of QD dopants to the beads requires that the beads' pores will not get saturated until the end of the doping process. If n_{MAX} is a maximum number of QDs per unit volume that can be embedded into the bead, than for the detection system shown in FIG. 5 the number of QDs of i-th type which are detected through i-th mirror will be

$${n}_{i} = 1 / {η}^{i} \times \frac{1 - η}{1 / {η}^{5} + 1 / {η}^{4} - 2} \times \frac{{n}_{MAX} \times ( 4 / 3 ) π {d}^{3}}{γ} \vert$$

where γ is a number of intensity gradations in each QD type, and n_{MAX} can be estimated as ˜3,700 beads/μm³. Thus, in our detection system minimum number of quantum dots of one color per bead N_{MIN} will be 58 and 912 for 2 μm and 5 μm beads correspondingly. If one quantum dot can produce φ=10-20 photons per ms per 1 mW of excitation power in optical system with 0.5% fluorescence collection/detection efficiency. Thus, the minimum number of photons detected from one bead

Φ_{MIN}=φ×N_{MIN}R_{LASER}/f_{CCD}.|

For laser power P_{LASER}=10 mW and f_{CCD}=1,000/s Φ_{MIN}=6,000-12,000 for 2 μm beads and increases up to 90,000-180,000 photons per frame for 5 μm beads.

The data acquired by CCD cameras is then copied to a single computer that performs color deconvolution and calls beads' signatures. Color deconvolution is required since quantum dots may have overlapping spectra. Color deconvolution is done the same way as in DNA sequencing utilizing a color matrix that is determined in advance for all types of quantum dots. In assigning individual signatures to the beads, we may want to use absolute values of fluorescent signals obtained in all color channels or we may want to utilize only ratios of intensities in different color channels (e.g. we will consider ratios 1:1:1:1:1:1:1:1:1 and 5:5:5:5:5:5:5:5:5 as the same signature). In case when we have Q≦9 types of quantum dots and γ gradations in each quantum dot type the number of unique signatures in the set of beads will be smaller by

F=(2×5^{Q}+3^{Q}+2^{Q+1}−7)/γ^{Q}|

fold. For our case when Q=9 and γ=10 we obtain F approximately 4%. Beads' calling will yield ˜10⁹ beads' signatures that have to be analyzed for uniqueness. This will be done using a refinement of the standard approach that utilizes a prefix tree data structure. Our estimates show that processing and analysis for uniqueness of the 10⁹ set of beads will take a few minutes if performed on a desktop computer of standard specifications.

### Example 4

### Manufacturing Beads with Nucleic Acid Markers

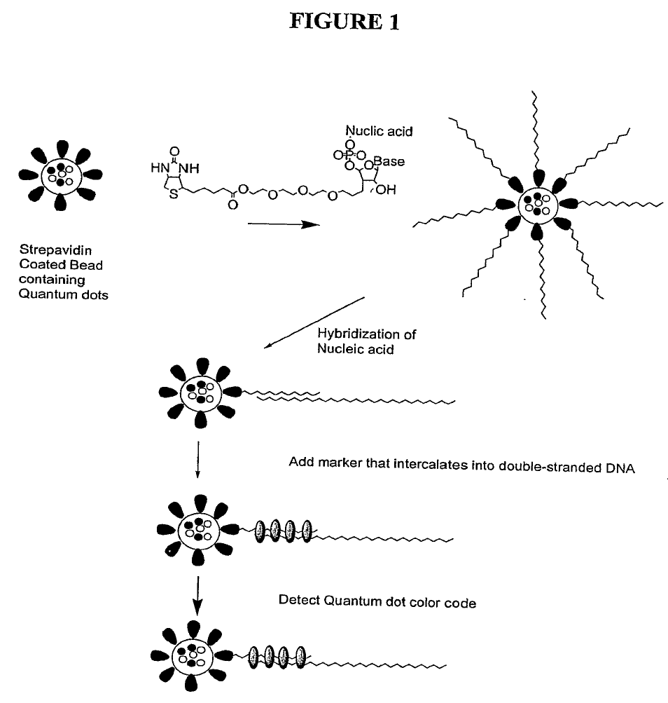

In some embodiment of the invention, the spectrally encoded beads may be used as general platform for detection of nucleic acid disease markers (see FIG. 1). For example, one produces a billion beads using the methods as described in Example 2. One coats the beads with streptavidin and binds them to biotinylated oligonucleotides (oligos). The oligos are sequences that hybridize to nucleic acid biological markers preferably disease markers.

Each well contains a single nucleic acid marker, and each nucleic acid contains a biotin moiety. For example, one amplifies 1,000 individual nucleic acid disease markers into 1,000 individual wells. One distributes the billion beads to each of wells containing the markers and streptavidin such that each bead contains a single sequence that corresponds to the disease marker. One passes the beads in each well through the detection system as described in Example 3, and one generates a computer file that identifies each code that is present on each bead and records the corresponding nucleic acid in the well. This is done for each bead in each well. One disregards, or considers on a statistical bases, beads with identical color coated markers such that confusion does not occur regarding the nucleic acid content of a particularly coded bead. Preferably only beads with a unique code correlate to a particular disease marker.

After recording the color codes of the beads, one mixes the entire one billion beads in a manner that distributes the 1,000 nucleic acid disease markers. One places a million of the billion mixed beads in a chamber. One exposes the chamber of mixed beads to a sample solution containing or suspected of containing a nucleic acid that hybridizes to the nucleic acid marker. One appropriately washes and collects the beads. One exposes the beads to a double helix intercalating agent such as ethidium bromide to determine hybridization. One analyzes the beads for color codes having a positive indication of hybridization and one correlates the presence or absense of a disease based on the correspond disease marker using the data from the computer file.

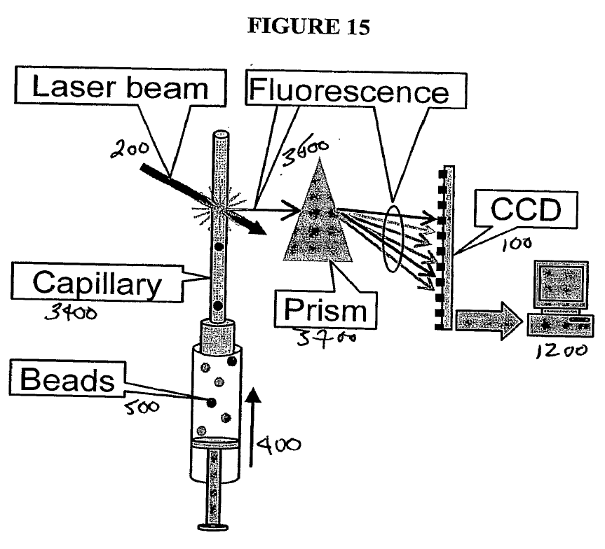

In a preferred embodiment, one prepares a large set of N beads which carry C_{U} unique spectral codes distinct (e.g. N≈3.6×10⁹ and C_{U}≈10⁹) diluted in a buffer and placed in a vessel, each bead carries a bio-molecule e.g. streptavidin) which allows binding DNA molecules. One prepares a well tray comprising w wells (e.g. w=1,000), each well contains multiple molecules of a genetic marker of one type, different genetic marker are in different wells. Genetic marker is a DNA or RNA fragment of a specific sequence. All genetic markers incorporate biomolecules which devise their binding to beads. One divides N beads evenly between w wells and incubate the well tray so that said molecular markers bind to said beads. One reads out spectral codes of all beads in each of w wells and put this information into a file (call it “PASSPORT” file). The PASPORT file contains codes of each individual bead and information about the type of the marker which this bead may carry. Readout of beads' codes can be done using a high throughput capillary bead detection system which is described elsewhere in this patent application. Said detection system has a provision for collecting all beads after the readout in a single vessel. One washes out said beads and dilutes them again in an appropriate buffer. One distribute all N beads over K vials so that each vial contained n≈N/K beads, among them approximately c_{U}≈C_{U}/K beads which carry unique codes. (e.g. if K=1,000 c_{U} will approximately be 10⁶ uniquely encoded beads per one vial which carry all w types of genetic markers, approximately 1,000 beads per each type of marker). One carries out an hybridization assay by placing test sample in a vial and incubating the vial at appropriate conditions (time, temperature, etc.). If hybridization happens for a specific marker type on a specific bead, the obtained double stranded DNA can be detected by labeling with fluorescence dye which specifically binds to double stranded DNA (e.g. SYBR green) or by any other known labeling technique which is used in hybridization assays. One washes out the beads with hybridized DNA and dilutes them in a vial with a fresh buffer. One reads out spectral codes of all said beads with hybridized DNA in said vial and put this information into a file (call it “VIAL” file). The VIAL file contains codes of each individual bead in said vial and information on hybridization on the bead (said information may include fluorescence intensity associated with the presence and the amount of the hybridized DNA, etc). Readout of beads' codes can be done using a single capillary reader (FIG. 15). One uses the appropriate software to compare codes of beads in the VIAL file with codes in the PASSPORT file and determine which specific genetic markers hybridized. One performs statistical analysis of the results obtained in the hybridization assay.

### Example 5 Manufacturing Monolith Multi-Capillary Arrays

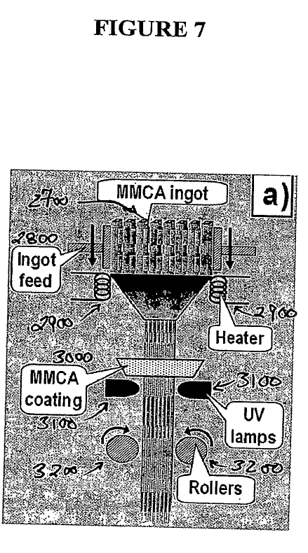

The process is illustrated in FIG. 7. One starts with a set of glass ferrules, their number equal to the desired number of channels. The size and the shape of the ferrules and the thickness of their walls are chosen depending on the desired inner size of the capillaries and the spacing between them. One presses the ferrules together in an array, packed in a glass tube of square shape, and drawns at an elevated temperature. After the drawing is completed, one cuts an entire capillary structure to provide MMCAs of the required lengths. Due to adhesion the resulting array has a monolithic structure. The production process allows formation of regular arrays of square or rectangular capillaries with translational symmetry. Significant advantages of the MMCA include the absence of any specially adjusted parts in the detection zone.

### Example 6

### Single-Molecule PCR on Microparticles in Water-in-Oil Emulsions

One may analyze a PCR product by agarose gel electrophoresis, and quantify the DNA yield using the PicoGreen dsDNA kit. Binding of primers to streptavidin-coated magnetic beads is done by suspending the beads in binding buffer and adding biotin conjugated primer. One may prepare emulsifier-oil mix using 7% (wt/vol) ABIL WE09, 20% (vol/vol) mineral oil and 73% (vol/vol) Tegosoft DEC and then vortex this mix. One may set up amplification reaction by mixing primers, template DNA, dNTPs, buffer polymerase, and water, and add, in order, one steel bead, oil-emulsifier mix and PCR mix to one well of a storage plate. One may seal the plate with an adhesive film and turn the plate upside down to make sure the steel bead moves freely in the well.

One may assemble a TissueLyser adaptor set (emulsions can also be generated using a stir-bar or a homogenizer) by sandwiching the 96-well storage plate containing the emulsion PCR mix between the top and bottom adapter plates, each fitted with a compression pad facing the 96-well storage plate and place the assembly into the TissueLyser holder, and close the handles tightly. When using less than 192 wells, balance TissueLyser with a second adaptor set of the same weight. One may mix once for 10 s at 15 Hz and once for 7 s at 17 Hz, temperature cycle the emulsions, and resuspend beads in 0.1 M NaOH and incubate for 2 min. One may place the tube in a magnetic separator for 1 min and carefully remove the supernatant. Detection of DNA on the beads can be done with fluorescent oligohybridization. One may use flow cytometry to determine the relative fluorescence intensity of the primers hybridized to the DNA on the beads.

The amount of DNA used in the emulsion PCR can vary over a relatively wide range. Optimally, 15% of the beads should contain PCR products. Using too little template results in too few positive beads, compromising the sensitivity of analysis. Using too much template results in too many compartments containing multiple templates, making it difficult to accurately quantify the fraction of initial templates containing the sequence of interest. Nonmagnetic beads can be used but centrifugation rather than magnets should be used to manipulate them.

The efficiency of amplification on solid supports in emulsions decreases with increasing amplicon length. The preferred amplicon length (including primers) is 7-110 bp. One may use a universal primer as the reverse primer. But one can also use a nested reverse primer, which yields an amplicon shorter than the product of the preamplification step to reduce nonspecific amplification on the beads or to decrease the size of the bead-bound PCR product. Use of higher polymerase concentrations results in higher yields of PCR products bound to beads. Another way to increase the amount of PCR product bound to the beads is through rolling circle amplification.

### Example 7

### Fabrication of Monolith Multi-Capillary Arrays

One starts with a set of glass ferrules. The size and the shape of the ferrules and the thickness of their walls are chosen depending on the desired inner size of the capillaries and the spacing between them. The ferrules are pressed together in an array, packed in a glass tube of square shape, and are drawn at an elevated temperature to melt them together. After the drawing is completed, an entire capillary structure can be cut to provide the required lengths. Due to adhesion, the resulting array has a monolithic structure. The production process allows formation of regular arrays of square or rectangular capillaries with a translational symmetry. Being monolithic, the array acts as a low-loss medium for the propagation of light.

One fabricates 32×32 and 64×128-capillary arrays with square 5 μm and 10 μm capillary cross sections and 10 μm and 15 μm array pitch. To prevent the abstraction of the labeled DNA on capillary walls, one uses a capillary wall coating such as BSA.

### Example 8

### PCR on Coded Beads

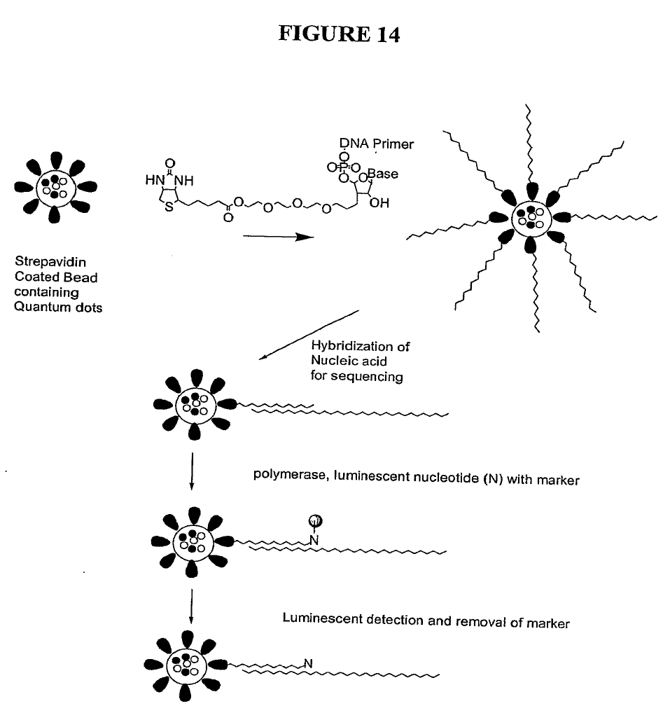

Beads covalently coated with streptavidin are bound to biotinylated oligonucleotides (oligos) (see FIG. 14). An aqueous mix containing all the necessary components for PCR plus primer-bound beads and template DNA are stirred together with an oil/detergent mix to create microemulsions. The aqueous compartments contain an average of less than one template molecule and less than one bead. The microemulsions are temperature-cycled as in a conventional PCR. If a DNA template and bead are present together in a single aqueous compartment, the bead-bound oligonucleotides act as primers for amplification.

### Example 9

### Document Authenticity

In some embodiments, the invention relates to a method of using a set of multi-colored beads to uniquely tag non-human-edible products. One (i.e. agency) first generates, as described above, a set of beads of size N, each one tagged by a different color combination (i.e., each bead has a different tag). From this set one sends N, M beads to the client who wishes to tag a number of products. One calls this the tagging set. The client then takes a small, measured part of the tagging set of beads he has, and dopes the each sample of the product he wishes to tag with the small, measured part (The number of beads in this set is T). The product is now tagged, and detection can then occur at any moment. A subset of beads in the product sample are separated, and read. Now, the detector has a set of tags. This set of tags is sent to the Agency, which then tells the detector who ordered the tagging set, and any information the purchaser of the tagging set wanted to disseminate to detectors.

One computes the relative proportions of M, N, and T, required to tag and then, with very high probability, uniquely determines the tagging set from a single sample. First, one introduces an additional factor, R: The fraction of the beads in the sample that were recovered for detection.

Assuming N total beads, an M-sized tagging set, T beads per sample, and T×R beads per detection attempt, one gets: The probability of a bead being in a different set, if it is in the tagging set, is: (M/N). The probability of T×R beads being in ONE different set is, as they are independent of each other: (M/N)^{(T×R)}, One calls this the collision probability.

To compute the probability of any 2 selections being the same, one refers to the birthday paradox. It states that for objects with collision probability of X, and K objects, the probability of no 2 being the same is roughly (1−X)^{C(K,2)}, which simplifies to (1−X)^{(1/2×K×(K-1))}

One wants to have K object with probability B that none are the same. For this, we have to solve (for K):

(1−X)^{(1/2×K×(K-1))}=B

For this, one gets:

K=(ln [1−X]+{[log [1−X]]}^{1/2}×{8×log [B]+ln [1−X]}^{1/2})/(2×ln [1−X])

Thus, for M=10⁸, N=10⁹, and R=1, the collision probability is 0.1^{T}. For T=20, the collision probability is thus 10⁻²⁰. If one wants the overall collision probability to be at most 0.95, we then can have up to 3×10⁹ distinct objects.

### Example 10 Illustrates Beads' Transfer in Electric Field

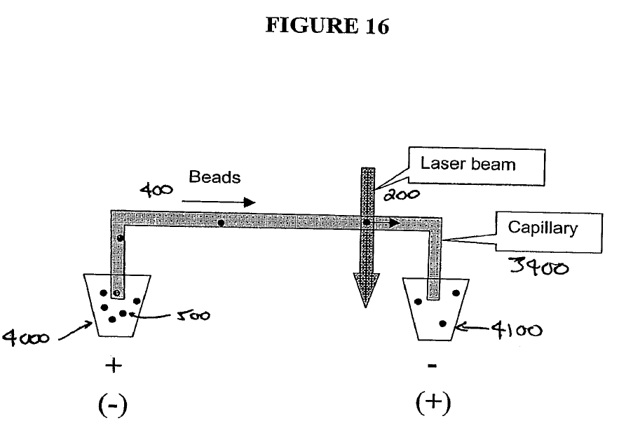

Two tubes (FIG. 16) which contain a buffer solution and comprise spectrally barcoded beads are connected by a single capillary or by a multi-capillary array. One uses carboxyl functionalized, 500 nm polystyrene divinylbenzene beads doped with Quantum dots (CrystalPlex Plex 890 William Pitt Way, Pittsburgh, Pa. 15238). Electric potential is applied between said tubes. If beads carry an electric charge they move along the capillary. Detection of the beads is done by using laser excited fluorescence.

1. A method of determining a phenotype of a subject comprising:
a) providing,
i) a plurality of linked beads wherein said beads comprise
A) luminescent electromagnetic codes,
B) a plurality of nucleic acid markers which hybridize to nucleic acids that correlate to a phenotype of a subject, and wherein said plurality of nucleic acids markers are configured such that nucleic acids with a unique sequence are linked to a bead with a unique luminescent electromagnetic code, and
ii) a sample containing or suspected of containing nucleic acid from said subject;
b) detecting said luminescent electromagnetic codes on said plurality of beads and recording said codes to correspond to said unique sequence of said nucleic acid marker on said beads;
c) mixing said linked beads with said sample under conditions such that hybridization to nucleic acids in said sample can occur,
d) detecting a bead where hybridization occurs;
e) determining said luminescent electromagnetic code on said hybridized bead;
f) comparing said luminescent electromagnetic code on said hybridized bead to said recorded codes; and
g) correlating said recorded code to said phenotype in said subject.

2. The method of claim 1, wherein said luminescent electromagnetic codes comprises more than three distinguishable electromagnetic wavelengths.

3. The method of claim 1, wherein said luminescent electromagnetic codes comprises more than ten distinguishable electromagnetic wavelengths.

4. The method of claim 1, wherein said electromagnetic wavelengths are discrete visual colors.

5. The method of claim 1, wherein number of said beads exceeds 1,000,000.

6. The method of claim 1, wherein said plurality of nucleic acid markers includes 1000 different markers.

7. The method of claim 1, wherein said beads are linked to said nucleic acids by a biotin-streptavidin interaction.

8. The method of claim 1, wherein said phenotype is a disease.

9. The method of claim 1, wherein said subject is a human.

10. A detection system comprising:
a) a first bead comprising a first luminescent label and a second luminescent label;
b) a second bead comprising a third luminescent label and a fourth luminescent label;
c) a first transparent channel capable of accepting said first bead and a second transparent channel capable of accepting said second bead; and
d) an instrument for detecting electromagnetic radiation from luminescent labels.

11. The system of claim 10, wherein said first and second luminescent labels are fluorescent quantum dots.

12. The system of claim 10, wherein said first luminescent label and said third luminescent label are the same label wherein said third luminescent label is at lower concentration per bead inside said second bead than is the concentration per bead of said first luminescent label inside said first bead.

13. The system of claim 10, wherein a common wall separates said first and second transparent channels

14. The system of claim 10, wherein said first and second transparent channels comprise a square cross section.

15. The system of claim 10, wherein said instrument for detecting electromagnetic radiation comprises a charge-coupled device.

16. The system of claim 10, further comprising a source of electromagnetic radiation.

17. The system of claim 16, wherein said source of electromagnetic radiation is a laser.

18. A method of generating luminescent encoded beads comprising:
a) providing:
i) a plurality of first luminescent particles,
ii) a plurality of second luminescent particles, and
iii) a plurality of porous structures;
iv) a first plurality of wells wherein
said first luminescent particles are in said wells and have different concentrations in at least two of the wells;
v) a second plurality of wells
wherein said second luminescent particles are in said wells and have different concentrations in at least two of the wells;
b) distributing a portion of said plurality of porous structures to said first plurality of wells under conditions such that said first luminescent particles are absorbed by said porous structures;
c) extracting said plurality of porous structures with said first luminescent particles from said first plurality of wells;
d) mixing said extracted plurality of porous structures with said first luminescent particles together to form a plurality of porous structures wherein at least two of said porous structures have different concentrations of said first luminescent particles;
e) distributing said plurality of porous structures wherein at least two of said porous structures have different concentrations of said first luminescent particles to said second plurality of wells under conditions such that said second luminescent particles are absorbed by said porous structures;
f) extracting said plurality of porous structures with said first luminescent particles and said second luminescent particles from said second plurality of wells
g) mixing said plurality of porous structures with said first luminescent particles and said second luminescent particles that are extracted from said second plurality of wells together to form a plurality of porous structures wherein at least two of said porous structures have different concentrations of said first luminescent particles and have different concentrations of said second luminescent particles.

19. The method of claim 18, wherein said first luminescent particle is a quantum dot.

20. The method of claim 18, wherein said second luminescent particle is a quantum dot wherein said first and second quantum dot have a different size.

21. The method of claim 18, wherein said porous structures are mesoporous silica beads.

22. The method of claim 18, wherein said porous structures are mesoporous polystyrene beads.

23. The method of claim 18, wherein said conditions for distributing a portion of said plurality of porous structures to said first plurality of wells does not saturate the porous structures with said first plurality of particles.

24. The method of claim 18, further providing a plurality of third luminescent particles, wherein said second and third luminescent particles are in said wells and have different concentrations in at least two of the wells and further mixing said plurality of porous structures with said first luminescent particles said second luminescent particles and said third luminescent particles that are extracted from said second plurality of wells together to form a plurality of porous structures wherein at least three of said porous structures have different combinations of concentrations of said first, said second and said third luminescent particles.

25. A method of determining the authenticity of an object comprising
a) providing
i) an object comprising plurality luminescent encoded beads,
wherein said encoded beads comprise two or more luminescent markers configured to provide a luminescent signature,
ii) electromagnetic radiation, and
iii) an instrument for detecting electromagnetic radiation;
b) placing said object in said electromagnetic radiation under conditions such that said quantum dots luminesce, and
c) detecting said luminescent signature with said instrument; and
d) correlating the luminescent signature with the authenticity of said object.

26. The method of claim 25, wherein said object is selected from the group consisting of a personal identification card, cash, liquid, solid, and fabric.

27. The method of claim 25, wherein said electromagnetic radiation is ultraviolet light.

28. The method of claim 25, wherein said luminescent markers are quantum dots.

29. A method of determining a DNA sequence comprising:
a) providing,
i) a plurality of linked beads wherein said beads comprise
A) luminescent electromagnetic codes,
B) a plurality of identical single stranded nucleic acid molecules
wherein said plurality of nucleic acids molecules are configured such that an individual bead having an individual code carries nucleic acids molecules with a unique sequence;
ii) a plurality of wells at least four of said wells contain solutions with four different fluorescently labeled nucleotides;
b) detecting said luminescent electromagnetic codes on said plurality of beads and recording said codes;
c) placing said linked beads into one of said wells containing one of said free nucleotides and incubating said beads under conditions such that said free nucleotides are attached to said nucleic acids molecules to form a complementary strand;
d) detecting said linked beads and determining said codes of said beads for which the incorporation of said free nucleotides occurs;
e) recording in a computer file said codes of said beads for which the incorporation of said free nucleotides occurs;
f) removing fluorescence label from said incorporated free nucleotides;
g) repeatedly repeating steps c), d), e), and f) for all said free nucleotides;
h) analyzing said recorded data on said incorporated nucleotides for each individual bead and determining sequence of said nucleic acid molecules linked to said individual bead.

30. A method of moving a bead through a channel comprising:
a) providing:
i) bead comprising a first luminescent label and a second luminescent label,
ii) a channel,
iii) a solution inside said channel wherein said beads are inside said solution,
iv) pair of electrodes; and
b) applying a potential between said pair of electrodes under conditions such that said bead moves in said channel toward one electrode of said electrode pair.

31. The method of claim 31, wherein said bead is a polystyrene bead.

32. The method of claim 31, wherein said first and second luminescent labels are quantum dots.

32. The method of claim 32, wherein said bead is charged.

33. The method of claim 32, wherein said bead has a carboxyl functionalized surface.
